(19) United States
(12) Patent Application Publication
(10) Pub. No.: US 2026/0335395 A1
(43) Pub. Date: Oct. 29, 2026

(54) CHINESE HERBAL FORMULATIONS AND USE THEREOF

(71) Applicants: Yuval Kaufman, Haifa (IL); Avraham Klein, Tel Aviv (IL); MORE RESEARCH APPLICATIONS LTD., Ramat Gan (IL)

(72) Inventors: Yuval Kaufman, Haifa (IL); Avraham KLEIN, Tel Aviv (IL)

(21) Appl. No.: 19/114,330

(22) PCT Filed: Sep. 21, 2023
(86) PCT No.: PCT/IB2023/059363

§ 371 Date: Mar. 21, 2025

Related U.S. Application Data
(60) Provisional application No. 63/376,510, filed on Sep. 21, 2022.

(51) Int. Cl. A61K 36/88; A61K 31/12; A61K 36/21; A61K 36/232; A61K 36/233; A61K 36/286; A61K 36/35; A61K 36/536; A61K 36/537; A61K 36/65; A61K 36/66; A61K 36/89; A61K 36/9066; A61P 15/00

(52) U.S. Cl. CPC A61K 36/88; A61K 31/12; A61K 36/21; A61K 36/232; A61K 36/233; A61K 36/286; A61K 36/35; A61K 36/536; A61K 36/537; A61K 36/65; A61K 36/66; A61K 36/89; A61K 36/9066; A61P 15/00

(57) ABSTRACT

Chinese herbal formulations comprising at least one herb or a component thereof from three or more different classes of Chinese herbs selected from Blood tonifying, Blood moderators, Blood heat coolers, body heat coolers and Yang tonic are provided. These herbal formulations are useful for treating pain, Blood stagnation, inflammatory and/or hormone mediated diseases, disorders and conditions, for example, endometriosis and other inflammatory and/or hormone mediated diseases of the pelvis and lower abdomen.

### FIELD OF THE INVENTION

The present disclosure relates to herbal formulations useful in preventing or reducing the severity of different inflammatory and/or hormone mediated pathologies, particularly, but not exclusively, diseases, disorders and conditions of the pelvis and abdomen.

### BACKGROUND

Traditional Chinese medicine (TCM) is a 3500-year-old medicine, based on the Taoist philosophy that originated in the far east, in ancient China in particular. The knowledge has passed from teachers to students, and over the years gathered in several manuscripts that serve us today in the modern way of teaching.

“Yin/Yang Theory” is a conceptual framework of TCM. According to the Yin/Yang theory, the universe is a result of a unity of opposing forces termed Yin and Yang. Yin and Yang, together, comprise the ‘one’ or the whole. The dynamic equilibrium between Yin and Yang determines the status/phase of a given subject.

Yin is a tangible quality and accounts for whatever has form, whatever can be touched or sensed with the physical senses. In other words: a matter. Yang is a non-tangible quality and accounts for whatever is untouchable. Yang is the potential of living, the vitality, the aspect that brings the living spark into matter/substances. Once Yang is deficient, physiological processes are impaired, and physiological systems start to develop pathologies.

Yin serves as the ‘fuel’ or the nourishing aspect of processes, the wood one throws into the fire for keeping it up. Yang provides the ability to transform matter into ‘life’. A living creature that lacks the Yin aspect will become weaker and thinner, with little ability to maintain necessary processes for daily life. The Yang will then be depleted, as Yin nourishes the Yang.

Traditional Chinese medicine views the human body as an organic entity, consisting of an assembly of various organs that function in a mutually interdependent manner. It classifies body structures, explains clinical symptoms, and guides treatment of diseases based on the Yin/Yang theory. Thus, functional activities of the body are classified as Yang, while the material basis (such as essence, blood, and body fluids) of vital functions are classified as Yin.

In TCM, the interaction between Yin and Yang generates Qi. Qi (pronounced “chee”) refers to the refined and nutritive substances flowing in the body as well as the functional status of organs and tissues. Within this framework, the complete deprivation of Qi signifies death in TCM.

In practice, oriental medicine, including TCM, includes both herbal remedies and acupuncture. Acupuncture is based on a meridional theory, which incorporates a system of channels through which vital energy (i.e., Qi) flows. One of the earliest descriptions of meridians and acupuncture points (acupoints), as well as technical aspects of acupuncture and moxibustion, was written by Hangfu Mi in 259-282 AD. This book describes 349 acupuncture points.

Herbal medicine (HM) has been proven to benefit millions of patients around the world afflicted and suffering from various conditions, disorders and/or diseases. Herbal medicine has been shown to be superior to placebo and to other medical treatments and is being used by many practitioners worldwide.

### SUMMARY

The present disclosure addresses the yet unmet need for specific HM-based remedies that provide clinically proven benefits in inflammatory and hormone mediated pathologies such as, but not limited to, endometriosis.

In one aspect, the present disclosure relates to herbal compositions, particularly but not exclusively, to easily digestible oral herbal compositions, for treating various inflammatory and/or hormone mediated pathologies such as, but not limited to, endometriosis, adenomyosis, chronic pelvic pain, irritable bowel syndrome, interstitial cystitis and painful bladder syndrome diseases. In some embodiments, the herbal oral compositions comprise traditional Chinese herbs.

A herbal formulation disclosed herein comprises at least one herb or a component thereof from three or more different classes of Chinese herbs selected from Blood tonifying, Blood moderators, Blood Heat coolers, body Heat coolers and Yang tonic herbs, wherein:
(i) the Blood tonifying herbs are selected from: Bai Shao, Dang Gui, Gou Qi Zi, He Shou Wu, Sang Shen Zi and Shu Di Huang;
(ii) the Blood moderating herbs are selected from Pu Huang, Yan Hu Suo, Hong Hua, San Leng, Dan Shen, Huai Niu Xi, Jiang Huang (curcumin), Ai Ye, Ce Bai Ye, Di Yu, Huai Hua Mi, Lian Fang, Ou Jie, Wang Bu Liu Xing, and Zong Lu Pi, Chi Shao, Chuan Niu Xi, Chuan Shan Jia, Chuan Xiong, Ding Jin Cao, E Zhu, Gan Qi, Hu Zhang, Ji Xue Teng. Liu Ji Nu, Ma Bian Cao, Qi Cao, Shui Zhi, Su Mu, Yi Mu Cao, Yu Jin, and Ze Lan;
(iii) the Blood or body Heat cooling herbs are selected from, Xia Ku Cao, Chai Hu, Mu Dan Pi, Cha Chi Huang, Ren Zhong Bai, Zhi Zi, Bo He, Man Ji Zhi, Mao ZI Dun Tou, Sang Ye, Yi Zhi Xiang, Di Gu Pi, Ji Guan Hua, Xi Jiao, Xuan Shen; Yin Chai Hu and Zi Cao; and
(iv) the Yang tonic herbs are selected from Xu Duan, Ba Ji Tian; Bu Gu Zhi; Du Zhong, Gou Ji, Hai Gou Shen, Hai Tao Ren; Jiu Zi; Rou Cong Rong, She Chuang Zi, Tu Si Zi and Xian Mao, and equivalents thereof.

For example, the in a disclosed herbal formulation (i) the Blood tonifying herb is Bai Shao, Dang Gui or both; (ii) the Blood moderating herbs are one or more of Pu Huang, Yan Hu Suo, Hong Hua, San Leng, Dan Shen, Huai Niu Xi and Jiang Huang (curcumin); (iii) the Blood or body Heat cooling herbs are one or more of Xia Ku Cao, Chai Hu, and Mu Dan Pi and (iv) the Yang tonic herb is Xu Duan.

In some embodiments, the herbal formulation comprises three or more herbs selected from: Bai Shao, Dang Gui, E Zhu, Pu Huang, Yan Hu Suo, Hong Hua, San Leng, Yi Mu Cao, Dan Shen, Huai Niu Xi, Jiang Huang, Xia Ku Cao, Chai Hu, Mu Dan Pi or Xu Duan, Du Zhong, Rou Cong Rong, Xian Mao, Yi Zhi Ren, Ba Ji Tian, or Qing Dai, wherein at least three herbs belong to at least three different classes.

The herbal formulation may be formulated as a solid dosage form such as capsule, pill, caplet, powder or a tablet, or as a liquid dosage form. Herbal formulation may be formulated as a single unit-dose dosage form or as a multiple unit-dose dosage suitable, for example, for oral administration.

In another aspect, the present disclosure relates to methods for treating, preventing and/or alleviating one or more of: pain, Blood stagnation, inflammatory and/or hormone mediated diseases, disorders and conditions, for example, inflammatory and/or hormone mediated diseases, disorders and conditions of the pelvis and lower abdomen. The methods described herein comprise administrating to a subject in need thereof of therapeutically effective amounts of one or more of the herbal formulations disclosed herein.

In some embodiments, the inflammatory and/or hormone mediated diseases, disorders and conditions of the pelvis and lower abdomen include, for example, endometriosis, adenomyosis, chronic pelvic pain, painful bladder syndrome, interstitial cystitis, inflammatory bowel disease, irritable bowel syndrome, vulvodynia, vestibulodinia (vestibulitis), myofascial syndrome and associated morbidity including chronic fatigue syndrome, premenstrual syndrome (PMS) symptoms mood changes and catamenia hemothorax.

In some embodiments, the disease treatable by a disclosed method is endometriosis.

### BRIEF DESCRIPTION OF THE DRAWINGS

Some embodiments are herein described, by way of example only, with reference to the accompanying drawings. With specific reference now to the drawings in detail, it is stressed that the particulars shown are by way of example and for purposes of illustrative discussion of embodiments. In this regard, the description taken with the drawings makes apparent to those skilled in the art how embodiments may be practiced.

In the drawings:

FIGS. 1A-1E are graphs showing the results of a clinical study evaluating the effects of treating endometriosis patients with an exemplary herbal formulation (Formulation (IIa)). The effect of treatment on percent of pain free days is shown. Pain-free days were evaluated with respect to bowel movement (1A), pelvic pain (2A), sexual intercourse (1C), lower abdominal pain (1D) and other pain (1E). Group A: study group (n=22); Group B: placebo group (control; n=17);

FIGS. 2A-2C are graphs showing the results of a clinical study evaluating the effects of treating endometriosis patients with an exemplary herbal formulation (Formulation (IIa)). The effect of treatment on pain experienced by the patients is shown. Total amount of pain was assessed with respect to back pain (2A), lower abdominal pain (2B) and thigh pain (2C). Group A: study group (n=22); Group B: placebo group (control; n=17); and

FIGS. 3A-3F are graphs showing the mean Endometriosis Health Profile Questionnaire (EHP-30) scores for a study group (n=20) vs. control group (n=17) in a clinical study of endometriosis patients treated with an exemplary herbal formulation (Formulation (IIa)). EHP scores were evaluated by averaging the scores of all patients in each group for each of the categories surveyed in the questionnaires. The categories shown are physical status (3A), emotional status (3B), intercourse status (3C), digestive status (3D), last 4 weeks status (in terms of general feeling) (3E), and physical pain (3F). Group A: study group; Group B: placebo group (control). The numbers on the X axis denote the time points in the clinical study: initiation of study or entery (0); 2 months or midterm visit (2); and 4 months after initiation or final visit (4).

### DETAILED DESCRIPTION

The present disclosure is based on a discovery by the present inventors that certain mixtures of herbs, particularly medicinal herbs applied in Chinese medicine (herein, collectively and interchangeably referred to as “Chinese herbs” or “medicinal Chinese herbs”) improved, alleviated and/or mitigated symptoms of certain pathologies of the pelvis and abdomen such as endometriosis and other inflammatory mediated pathologies.

The present inventors hypothesized that specific, designated combinations of medicinal Chinese herbs from at least three classes (classified according to traditional Chinese classification/categories), will enhance the immune system and bolster the natural defense of the human body. Without being limited or held by a certain mechanism of action, the working hypothesis of the present inventors was that the application of such combinations of Chinese herbs in the treatment of pathologies associated with blood flow impairment, will improve blood flow to stagnant tissues (the blood is or may be normal but the flow of blood to these tissues is reduced or unevenly distributed) and, in addition, enhance or strengthen the body's natural defense.

For example, it was envisioned that provision of a contemplated Chinese herbal combination to women afflicted with endometriosis will improve blood flow to the stagnant tissues, dissolve small lumps and improve the selectivity of the immune system in affecting an ectopic tissue instead of causing inflammation of an already accumulated ectopic tissue.

The present inventors successfully produced herbal formulations comprising mixtures of medicinal Chinese herbs of different classes, designed to enhance ‘blood moderation’ (movement) along with blood and functional system enrichment. Exemplary formulations are described in Examples 1 and 2 herein. In Example 3, the capacity of a disclosed formulation (Formulation (IIa)) to improve pain and overall feeling and quality of life of endometriosis patients is demonstrated.

The blood is a carrier of oxygen, nutrients and other physiologically important substances. When blood flow to a specific part of the body is reduced, that part will not receive essential nutrients, and the symptoms of poor circulation may be experienced. For example, deficient blood flow may result in worsening of some symptoms of certain diseases or conditions and/or worsening of clinical pathologies. The term “Blood Deficiency” or “XUE XU”, as understood in East Asian Medicine (also interchangeably referred to herein as “traditional Chinese medicine” (TCM)) and as used herein, refers to an inability of the Blood of the body to perform its functions.

The “Chinese organs” do not represent the organs that share the same anatomical names in Western science and medicine. Herein, “Chinese “organs” are designated by a capital letter, e.g., Liver, Heart, Kidney, and the like. Likewise, the capitalized “Blood” is used herein to differentiate it from “blood”, the liquid that delivers oxygen to organs and tissues.

In East Asian Medicine, Blood has many functions, such as, but not limited to, circulating vital substances throughout the body (alongside Qi), including warmth and nutrients; providing nourishment to the Heart to allow for restful sleep and a calm mind; nourishing the brain and the Spleen (responsible for memory) to aid in learning; moistening the skin, nails, and hair; creating breast milk and sweat; providing the substance menstruation and gestation; bringing fluids to the muscles to allow for relaxation; and supporting the “Shen”, or consciousness.

Thus, the Blood is responsible for many vital functions in the body. A deficiency of Blood can manifest as the inability of Blood to perform any of the functions listed above, and therefore the symptoms of Blood Deficiency can vary from person to person. However, very common symptoms of Blood Deficiency are insomnia, muscle pain and tension, forgetfulness, anxiety and depression, fatigue, menstrual dysregulation, complications of pregnancy or postpartum, and insufficient lactation, dry skin, hair, and nails; a pale or lackluster complexion, blurry vision, and fainting or dizziness.

An acupuncturist or East Asian Medicine practitioner can diagnose Blood Deficiency using diagnostic tools such as feeling the pulses in the wrists, looking at the tongue, examining the lower eyelid or gums, and considering the patient's symptoms. These tools allow the provider to assess the quantity and functionality of Blood in the body, and to determine any co-pathologies that might be leading to Blood Deficiency.

By conferring Blood moderation and Blood enrichment to deficient Blood, the present inventors successfully achieved a co-balance of Blood (and blood) properties, contents and functions. This achievement is critically important since it is known in the art of TCM that obtaining a co-balance of Blood quantity and Blood quality is critical: deficiency in Blood content (not necessarily as defined in Western medicine) is usually treated according to TCM by acupuncture, targeting organs of the digestive system (stomach, small intestine, colon), and use of “Blood strengthening” herbs. However, if the quality of Blood remains poor, such treatment will fail to accomplish its goals. Moreover, supporting one aspect of Blood properties while failing to obtain the right balance with other aspects might lead to adverse side effects. Thus, the key for an effective herbal formulation is its ability to afford adequate levels of Blood quality, e.g., flow and the ability to regulate heat/cold, that is co-balanced with its ability to restore Blood quantity.

According to TCM, substances and energy are distributed throughout the body by the meridian pathways. A meridian is commonly defined as an energetic highway, road or channel in the human body. Meridians exist in corresponding pairs and each meridian has multiple acupuncture points along the pathway. The meridians are divided into the following main groups:
(a) Twelve (12) regular meridians (Mai), also termed “principal meridians”; and
(b) Eight (8) extraordinary/special meridians, also termed herein “8 extra”, (CV, GV, CHONG, Dai, Yin Wei, Yang Wei, Yin Qiao and Yang Qiao).

The 12 meridians run from head to toe along the body. Each of the 12 regular meridians is associated with a Chinese “organ”, such as the Heart, Pericardium, Lung, Spleen, Liver, Kidney (the Yin organs), and Stomach, Gallbladder, Large and Small Intestine, Urinary Bladder and tri-heater or triple burner (the Yang organs). Couples of the Yang meridians are associated together so as to form 3 physiological systems, and so do the Yin meridian couples, with each system being responsible for a specific role in the physiology of the body. For example, the JUE YIN system that consists of the GAN (corresponding to the liver though it is not the actual organ) and the XIN BAO (corresponding to the pericardium) is, among other things, responsible for controlling the blood flow.

The terms “Kidney Yang” and “Kid Yang”, each refers to one of the two energetic aspects of the Kidney. While each of the regular meridians is associated either with Yin or Yang, the Kidney is the only entity which has dual quality: it has both Kidney Yin and Kidney Yang.

Kidney Yang is considered the most basic and profound source of Yang energy. It serves as the ultimate source of body energy, and the foundation of the energetical structure of the human body physiology. Kid Yang is the stimulant of processes and the driver of balanced physiology. Once deficient, let alone depleted, processes will deviate from the correct mode of action and illness will develop.

Out of the 8 extraordinary meridians, 2 extra meridians have their own set of acupuncture cavities/points, the CV (front) and GV (back), and the other extra meridians are triggered/operated through points along some of the 12 regular meridians set. The extraordinary meridians are not associated with the major organ systems. These meridians have very specific functions. They act as reservoirs for Qi and blood. They circulate “substances” around the body, as they have a strong connection to the Kidneys. They help circulate Wei Qi, which acts as the immune system, and they provide connections between the 12 regular channels.

The 8-extra meridians are considered as highways of the energetic infrastructure of physiology, whereas the regular meridians, are considered as “regular roads” for energy routing.

To provide vital energy for supporting life activities, Qi flows through the meridians and nourishes the organs. With respect to its role in modulating physiological functions, Qi is subcategorized into three functionally related types: primordial Qi, pectoral Qi, and normal Qi. In essence, primordial Qi, which is also known as “congenital essence of the Kidney”, is inherited from parents and is responsible for stimulating growth and development as well as invigorating the vital activities of organs in the body; that is, it is Yang in nature. Pectoral Qi is comprised of the “natural air” inhaled by the Lungs and the “grain Qi” transformed from food and water by the Spleen and Stomach; that is, it is Yin in nature. The principal actions of pectoral Qi are to facilitate gas exchange in the lungs and regulate blood circulation in the heart as well as its rate of beating. Primordial Qi combines with pectoral Qi to form normal Qi (called Zheng Qi in Chinese; herein simply referred to as “Qi”), which circulates in the body for supporting various body functions. The interrelationship between primordial Qi and pectoral Qi is consistent with the notion that Qi arises from an interaction between Yin and Yang.

Normal Qi manifests as two functions: nutritive Qi and defensive Qi. While nutritive Qi nourishes the internal organs to sustain the physiological functions of the body, defensive Qi protects the body against disease-causing internal (e.g., inflammation and cancer) and external (e.g., bacteria and viruses) factors.

When the Qi flows freely, the body remains healthy. However, due to various causes like stress, the body's meridians can become clogged or stagnant. When a blockage occurs along the meridians, illness can take hold. Acupuncture and herbal formulations are ways to unclog the meridians. The Chinese medicine (CM) practitioner's purpose is regulating the specific Yin-Yang system that deviated from its desired function and rebalance it and restore natural order and functionality.

### Chinese Herbal Formulations

The present inventors prepared herbal formulations, which feature the use of several categories/classes of Chinese herbs such as, but not limited to, Blood moderators, bleeding stoppers, Blood tonic herbs, Blood/body heat coolers, and/or Yang tonic herbs. Such formulations provided multiple beneficial effects. The provision of these herbal combinations may be effective in treating many disorders and diseases, including, but not limited to, endometriosis, adenomyosis, chronic pelvic pain, painful bladder syndrome, interstitial cystitis, inflammatory bowel disease, irritable bowel syndrome, vulvodynia, vestibulodinia (vestibulitis), myofascial syndrome and associated morbidity including chronic fatigue syndrome, mood changes and catamenia hemothorax.

For example, Blood moderators and bleeding stoppers along with Blood/body heat coolers performed the required action of relieving or mitigating inflammatory symptoms, especially in the LIV, CHONG and XUE aspects of some pathologies treated.

The terms “LIV” and “GAN”, as used herein, are interchangeable and refer to the Liver meridian, which influences and is influenced by the Liver. In TCM, LIV (GAN) is responsible for the free flow and movement of the Blood (“XUE”) throughout the body and tissues. The Liver meridian is also associated with mucus tissues functionality and is therefore most relevant to the endometrium and other gynecologically related tissues.

“CHONG”, as used herein, is an extraordinary meridian that is tightly associated with Liver functionality and, among other roles, governs the pelvis and abdomen, and is related to many gynecological functions. In addition, CHONG is very much related to Blood (XUE) flow (quality) and quantity.

The Yang Ming system (which consists of the Stomach and Colon meridians) is the only system that is rich in both XUE (blood) and Qi. The present inventors noticed that irregularities in this system are very common in endometriosis and other Blood (quantity and quality) related pathologies. Therefore, at least some contemplated herbal formulations disclosed herein are specially designed to target the Yang Ming system for the purpose of treating pelvic diseases. For example, inclusion of Blood heat coolers and/or body heat coolers in a contemplated combination of Chinese herbs was found essential for the cooling action of the Yang Ming system.

Herbal formulations prepared and applied by the present inventors, in which at least Blood moderators and Blood tonifying herbs or components thereof were combined, successfully enabled a desired co-balancing, and were highly effective in strengthening the core aspect and stasis break down, which are the fundamental problems in inflammation in general and endometriosis in particular. Example 3 herein demonstrates, using the means of a Western clinical study, the successful outcomes of treating endometriosis patients with a disclosed herbal formulation (Formulation (IIa), taught in Example 2 herein).

### Chinese Herbs Classification or Categories

Chinese herbs are an enormous part of traditional Chinese medicine and have since ancient times been classified according to their basic actions on the human body. Their classifications are constructed based on the fundamental principles of Taoist philosophy upon which Chinese medicine itself is built.

Unlike conventional medicine, herbs in Chinese medicine aren't standard for everyone even if they have excellent health properties. This is because herbs that effectively address some specific needs of one individual may actually harm another individual who does not have these specific needs. For instance, provision of herbs that reduce and clear Internal Heat to a patient who has Internal Cold and exhibits a few cold symptoms, would actually harm the patient and worsen his condition, even though these herbs contain certain beneficial nutrients.

Embodiments described herein relate to Chinese herbal formulations prepared from combinations of herbs that are classified/categorized into the following non-limiting main classes/categories: Blood tonic, Blood moderators, Blood heat coolers, body heat coolers, bleeding stoppers and Yang tonic herbs.

### Blood Tonic Herbs

The term “Blood tonic herbs”, as used herein, refers to herbs which nourish, refresh and vitalize the Blood. The term “Blood tonic” is synonym with “Blood refreshing”, “Blood vitalizing” and “Blood nourishing”.

Blood tonic herbs assist in building healthy red blood cells and boost up the immune system. Blood tonic herbs used in embodiments of the present disclosure include, but are not limited to, Bai Shao, Dang Gui, Gou Qi Zi, He Shou Wu, Sang Shen Zi and Shu Di Huang.

Bai Shao (White Peony root) is the peeled root of the perennial herbaceous plants Paeonia lactiflora of the family Buttercup. It grows in Zhejiang, Anhui and Sichuan provinces in China. It is plucked in summer and autumn, peeled, boiled, and dried in the sun. Its main actions include tonify Blood, emolliate Liver to alleviate pain, calm and suppress Liver Yang. As such, it is useful in the treatment of indications/syndromes such as Blood Deficiency, sweating due to Blood Deficiency, hypochondriac pain, abdominal pain and muscular contracture pain of four limbs. Bai Shao has marked sedative and suppressant effects on the central nervous system (CNS).

This is a common herb applied for regulating menstruation, and it is often indicated for irregular menstruation. Other indications and syndromes that may benefit from treatment with Bai Shao, usually in combination with other herbs, include Liver Blood Deficiency with menstrual dysfunction; vaginal discharge and uterine bleeding accompanied by a pale face; lusterless nails; anemia; breast distention and premenstrual syndrome; diarrhea, borborygmus and abdominal pain.

Yet further effects attributed to Bai Shao include inhibiting secretion of gastric acid, which may be beneficious in treating peptic ulcers; antibiotic; antipyretic and anti-inflammatory effects; antiplatelet; cardiovascular: Bai Shao demonstrates a marked action to relax blood vessels, dilate peripheral blood vessels, and cause a slight decrease in blood pressure; and antioxidant.

Bai Shao tastes bitter and sour, it may be provided at a dose of 5-30 g (tincture (solution): 2-4 ml). Entering meridians: Liver, Spleen.

Blood Tonic herbs which can substitute or be alternatives to Bai Shao in herbal formulation disclosed herein include but are not limited to: Gou Qi Zi (Fructus Lycii); He Shou Wu (Radix, Polygony Multiflora); Sang Shen Zi (Mori Albae, Fructus); and Shu Di Huang (Rhemanniae Glutinosae Conquitae, Radix).

Dang Gui (botanical name: Angelicae Sinensis Radix; Latin: Angelica sinensis; Pin Yin: Dang Gui): a superior Blood tonic and one of the most used herbs in the Chinese herbal system. Dang Gui grows in Asia (e.g., in a specific region of northwestern China). The fresh roots are processed and used. Dang Gui extract has a very characteristic fragrance due to the presence of n-butylidenephthalide and ligustilide in the volatile oil.

Dang Gui is utilized for supporting healthy blood circulation, especially in the abdomen and pelvic basin, as well as to establish, support and maintain healthy menstrual balance in women. It also has analgesic and mild sedative (calming, relaxing) actions.

Dang Gui's further main pharmacological effects include:
(i) Effects on the uterus: administration of Dang Gui essential oil both stimulates and inhibits effects on the smooth muscle of the uterus. When the uterus is in a state of relaxation, Dang Gui induces contraction, whereas if the uterus is in a contracted state, it promotes relaxation. This dual action is credited for the therapeutic effect of relieving spasms and stopping pain.
(ii) Cardiovascular: improves overall blood circulation by decreasing whole blood viscosity or improving the hemorheological changes in Blood stagnation. It has an antiarrhythmic effect. Administration of Dang Gui is associated with reduction of plasma cholesterol and triglyceride levels, and a decreased risk of atherosclerosis.
(iii) Antiplatelet effect, like that of aspirin. Dang Gui inhibits thrombus formation.
(iv) Immunostimulant via inducing increased phagocytic activity of macrophages.
(v) Hepatoprotection via promoting the generation and protection of hepatocytes.
(vi) Antibiotic activity at least against Salmonella typhi, E. coli, Corynebacterium diphtheriae, Vibrio cholerae, Alfa-hemolytic streptococcus and Beta-hemolytic streptococcus.
(vii) Analgesic and anti-inflammatory extracts, assumingly by decreasing vascular permeability thereby reducing inflammation.

Dang Gui is primarily known as a “women's herb,” as it is effective in balancing women's hormone levels, both restraining and supplementing the body's production of estrogen as needed. It is for treating menstrual and menopausal symptoms, including migraine, cramps, mood fluctuations, and hot flashes.

Dang Gui tastes sweet, acrid or bitter and may be provided warm at a dosage of 3-15 g (tincure (solution): 2-4 ml). Entering meridians: Heart, Liver, Spleen.

In some embodiments, Dang Gui is used as an active ingredient in contemplated formulations due to its anti-inflammatory and sedative properties and its ability to regulate contraction of the uterus muscle. Particularly, Dang Gui provides the formulations with anti-inflammatory effect, and analgesic effect which is twice as that of acetylsalicylic acid.

Blood Tonic herbs, which can substitute or be alternatives to Dang Gui in herbal formulations disclosed herein include but are not limited to: Gou Qi Zi; He Shou Wu; Sang Shen Zi; and Shu Di Huang.

### Blood Moderator Herbs

The term “Blood moderator herbs”, as used herein, refers to herbs which balance and harmonize Blood constituents, and help in replenishing deficient Blood. Blood moderator herbs used in embodiments of the present disclosure include, but are not limited to: Pu Huang, Yan Hu Suo, Hong Hua, San Leng, Dan Shen, Huai Niu Xi, Jiang Huang (curcumin), Ai Ye; Ce Bai Ye; Di Yu; Huai Hua Mi; Lian Fang; Ou Jie; Wang Bu Liu Xing; Zong Lu Pi; Chi Shao; Chuan Niu Xi; Chuan Shan Jia; Chuan Xiong; Ding Jn Cao; E Zhu; Gan Qi; Hu Zhang; Huai Niu Xi; Ji Xue Teng; Liu Ji Nu; Ma Bian Cao; Qi Cao; Shui Zhi; Su Mu; Yi Mu Cao; Yu Jin; and Ze Lan.

Pu Huang (Common Name: Cat Tail Pollen, Cattail Pollen, Bulrush Pollen, Pollen Typhae, Botanical Name: Typha angustifolia pollen, Typha orientailis, Typha latifolia. Chinese/Pin Yin Name: Pu Huang): a traditional Chinese medicinal herb useful for resolving stasis, stop bleeding and promote diuresis. Pu Huang benefits the Blood and skin health. It has antiseptic, analgesic, anti-inflammatory, diuretic and astringent effects.

Pu Huang's further main pharmacological effects include:
(i) Hemostatic: water or alcohol extracts of Pu Huang may reduce bleeding time. Charred Pu Huang has a better hemostatic effect compared to that of unprocessed Pu Huang.
(ii) Cardiovascular: Pu Huang is associated with increased contractility of the heart, increased blood perfusion to the coronary arteries, decreased blood pressure, and decreased peripheral vascular resistances.
(iii) Antihyperlipidemic: elevates high density lipoprotein (HDL) and lowers total cholesterol (TC) levels, probably via decreasing absorption and increasing utilization of cholesterol.
(iv) Stimulating effects on the uterus at small dosages. At larger dosages, over stimulation may lead to spasms and cramps.
(v) Gastrointestinal: Pu Huang extract provides increased intestinal peristalsis and relieved intestinal cramps. Water extract of Pu Huang is effective for treatment of peptic ulcer and enteritis.
(vi) Anti-inflammatory effect, especially in treatment of burns and arthritis (assumingly by improving circulation and decreasing permeability of blood vessels).
(vii) Immunologic effects on both cellular and humoral immunity. At small dosages, Pu Huang has little or no effect; at moderate dosages, it inhibits the immune system; and at large dosages, it stimulates the immune system. Its immune-enhancing effect is associated with increased phagocytic activity of macrophages.

In some embodiments, Pu Huang is used as an active ingredient in disclosed formulations due to its anti-stagnative and anti-inflammatory properties, and its ability to regulate contraction of the uterus. Particularly, Pu Huang provides to the formulations disclosed herein qualities such the ability to reduce uterus bleeding and suppress inflammatory processes, which alleviate the symptoms of pathologies such as endometriosis. In addition, due to Pu Huang's ability to affect the immune system, formulations comprising same support macrophages activity, thereby increasing the effectiveness of the immune system in recognizing ectopic tissue before it proliferates. In addition, due to its effect on the peristaltic activity of gastro-intestinal system, disclosed formulations comprising Pu Huang provide abdominal pain relief.

Pu Huang Tastes Sweet and Acrid. Entering Meridians: Liver, Heart, Spleen

Blood moderator herbs, which can substitute or be alternatives to Pu Huang in herbal formulation disclosed herein include, but are not limited to: Ai Ye (Folium Artemisiae Argyi); Ce Bai Ye (Biotae Orientalis Cacumen); Di Yu (Radix Sanguisorbae Officinalis); Huai Hua Mi (Flos, Sophorae Japonicae Immaturus); Lian Fang (Receptaculum Nelumbinis); Ou Jie (Nodus Nelumbinis Nuciferae Rhizomatis); Wang Bu Liu Xing (Vacca Segetalis, Semenriae); and Zong Lu Pi (Fibra Stipulae Trachycarpi).

Yan Hu Suo (Common Name: Corydalis Rhizome; Botanical Name: Corydalis yanhusuo rhizome; Other Names: Chinese Poppy, Rhizoma Corydalis Yanhusuo; Pin Yin Name: Yan Hu Suo): invigorate the blood and promotes movement of Qi. This herb is a natural analgesic that has the approximately 100 times more pain-relieving effects than most opioid drugs, but without the side effects. Corydalis is a distant cousin of the poppy plant but without addictive properties. Several alkaloids have been isolated from the tuber of Yan Hu Suo, including corydaline, tetrahydropalmatine (THP) and dl-THP. Of the full range of 20 alkaloids found in the plant, THP is considered the most potent one, possessing analgesic activity, as well as exerting sedative-tranquillizing and hypnotic actions. The analgesic effect of electro-acupuncture combined with Yan Hu Suo, increases significantly. The herb alleviates pain caused by cold and any type of pain affecting the chest, abdomen or limbs including pain caused by Blood stagnation, Qi Stagnation and traumatic injury. The sedative and tranquilizing effects are attributed to its effects on the CNS.

Yan Hu Suo's further main pharmacological effects include:
(i) Anti-inflammatory effect in both acute and chronic phases of inflammation, probably by inhibiting histamine secretion.
(ii) Antiulcer effect via suppressing the secretion of gastric acid.
(iii) Cardiovascular effect assumingly via decreasing resistance of blood flow and increasing blood perfusion to coronary arteries. Yan Hu Suo decreases oxygen consumption of the cardiac muscle and increases contractility of the heart.
(iv) Adrenocortical effect, assumed by increasing hormone secretion from adrenal glands.
(v) Muscle relaxant effects.

Yan Hu Sou tastes acrid bitter. Entering Meridians: Heart, Liver, Stomach, Lung, Spleen, Pericardium. Dosage: 3-12 g. Maximum dosage: 20 g. Tincture: 2-4 ml.

Blood moderator herbs, which can substitute or be alternatives to Yan Hu Sou in herbal formulation disclosed herein include, but are not limited to: Chi Shao (Radix Paeoniae Rubrae); Chuan Niu Xi (Radix Cyathulae Officinalis); Chuan Shan Jia (Squama Manitis Pentadactylae); Chuan Xiong (Radix Ligustici Chuanxiong); Ding in Cao (Herba Linderniae); E Zhu (Rhizoma Curcumae Ezhu); Gan Qi (Lacca Sinica Exsiccatae); Hu Zhang (Rhizoma Polygoni Cuspidati); Huai Niu Xi (Radix, Achyranthis Bidentatae); Ji Xue Teng (Radix Et Caulis, Jixueteng); Liu Ji Nu (Herba Artemisiae Anomalae); Ma Bian Cao (Herba Verbenae); Qi Cao (Holotrichia); Shui Zhi (Hirudo Seu Whitmania); Su Mu (Lignum Sappan); Yi Mu Cao (Leonuri Heterophylli, Herba); Yu in (Curcumae, Tuber); and Ze Lan (Lycopi Lucidi, Herba).

Hong Hua (Common Name: Flos Carthami; Other Names: Safflower, Saffron, Carthamus; Pin Yin Name: Hong Hua): herb that invigorates the blood. Useful in painful menstrual problems, post-partum hemorrhage and osteoporosis. Hong Hua has antioxidant, analgesic, anti-inflammatory and antidiabetic activities. It can significantly inhibit cell proliferation in a dose dependent manner, via apoptosis. Main indications: blood stasis, especially in women with gynecological problems such as abdominal pain, amenorrhea, postpartum dizziness, retained lochia and abdominal masses.

Hong Hua's further main pharmacologic effects include:
(i) Cardiovascular effect: a small dose of Hong Hua decoction has positive inotropic effects as it stimulates the heart, whereas a large dose has negative inotropic and chronotropic effects as it inhibits the heart and decreases overall cardiac output.
(ii) CNS sedative effects and preventative effects against drug-induced seizures.
(iii) Uterine stimulating effect via increasing uterus smooth muscle contraction, which may lead to decreased bleeding and dysmenorrhea.

Hong Hua tastes acrid (slightly bitter), sweet. Entering meridians: Heart, Liver, Lung. Dosage: 3-10 g (12-15 g=a large dose); 0.9-1.5 g when harmonizing Blood. Tincture: 2-4 ml.

Blood moderator herbs, which can substitute or be alternatives to Hong Hua in disclosed herbal formulations, are the same as those that can be alternatives for Yan Hu Sou, as disclosed herein.

San Leng (Common Name: Rhizoma Sparganii; Other Names: Burr-Reed Rhizome, Bulrush Rhizome, Scirpus, Sparganium Rhizome, Common Burr-Reed; Botanical Name: Sparganium stoloniferum; Rhizoma; Pin Yin Name: San Leng): herb that invigorates the Blood. Breaks blood stasis, regulates menses, regulates Qi and Qi stagnation, and alleviates pain. San Leng supports a healthy digestive system by dissolving accumulations (food stagnation). San Leng has a stimulating effect on the smooth muscle of the intestines, leading to muscle contractions. San Leng has anti-inflammatory, analgesic and sedative effects. It promotes lactation. Main indications/symptoms treatable with San Leng include blood stasis with amenorrhea, dysmenorrhea, postpartum abdominal pain and abdominal masses with severe abdominal pain and distention, menstrual irregularities and agalactia.

San Leng's further main pharmacologic effects include:
(i) Antineoplastic, inhibitory effect on cancer cells.
(ii) Hematological: effective in inhibiting aggregation of platelets and prolong thrombin time.

San Leng tastes bitter, acrid. Entering Meridians: Liver, Spleen. Dosage: 3-10 g. Tincture: 2-4 ml.

Dan Shen (Common Name: Salvia miltiorrhiza; Other Names: Chinese salvia, Chinese sage, red-rooted sage, tan shen; Pin Yin Name: Dan Shen): a native plant to Vietnam and China, its root is used for medical purposes alone or in combination with other medicinal plants for a variety of ailments, including heart diseases (e.g., angina pectoris), kidney diseases, irregular menstruation and other related problems (amenorrhea, dysmenorrhea), insomnia, irritability, inflammation, and Blood stagnation (circulatory problems). Dan Shen administration is associated with negative chronotropic and inotropic effects, and reduction of blood pressure. The herb dilates and increases blood perfusion to the coronary arteries to reduce the damage of cardiac ischemia and prevent or diminish death of cardiac muscle.

The constituents found in the root have adaptogenic actions to various types of stress. They prevent or treat osteoporosis. Dan Shen invigorates and nourishes the Blood and dispels Blood stasis; clears Heat and soothes irritability and reduces abscesses. Main indications/symptoms treatable with Dan Shen include Blood stasis in the Lower Jiao (very important in gynecology); Blood stasis obstructing the chest; rib and hypochondriac pain due to liver Qi and Blood stasis; Yin Stage Heat with restlessness, irritability, palpitations and insomnia; heart and kidney Yin Deficiency; and palpitations and insomnia due to Ying and XUE Stage Heat or heart Blood Deficiency.

More than 100 phytochemical compounds have been isolated from this plant. The major lipid-soluble phytochemical constituents of Dan shen are called tanshinones. The three primary bioactive tanshinones include tanshinone I (TNI), tanshinone IlA (TNIIA), and cryptotanshinone (CPT). These compounds exhibit anti-inflammatory, anticancer, and cardio-cerebrovascular protective actions.

Dan Shen's further main pharmacological effects include:
(i) Antiplatelet effects, which may last approximately 2 hours.
(ii) Anticoagulant and thrombolytic.
(iii) Antibiotic: it has an inhibitory influence on Staphylococcus aureus.
(iv) Hepatoprotective effect by lowering liver enzyme levels. Beneficial in treating liver cirrhosis. The hepatoprotective function is due, in part, to improving blood circulation and promoting regeneration of liver cells.
(v) CNS suppressant: Dan Shen has a marked sedative effect. It prolongs the hypnotic influence of phenobarbital and negates the stimulating effects of caffeine.

Dan Shen tastes bitter. Entering meridians: Heart, Pericardium, Liver. Dosage: 3-15 g, 15-30 g=large dose.

Blood moderator herbs, which can substitute or be alternatives to Dan Shen in herbal formulations disclosed herein, are the same as those that can substitute Yan Hu Sou.

Huai Niu Xi (Common/botanical Name: Achyranthes Japonica; Pharmacological Names: Radix Achyranthis Bidentatae, Bidentatae; other names: Achyranthes Root White Oxknee Root; Pin Yin Name: Huai Niu Xi): a herb that invigorates the blood and expels Blood stasis. Other actions of Huai Niu Xi include nourishing liver and kidney Yin, strengthening the sinews and bones and benefit the joints; descending Blood and Fire; clearing damp-heat in the Lower Jiao. Main indications/symptoms treatable with Huai Niu Xi include: blood stasis with dysmenorrhea, amenorrhea and retained lochia; pain due to trauma; low back and knee pain and soreness due to Kidney and Liver deficiency; Blood Heat in the upper Jiao with epistaxis, hematemesis and bleeding gums; Yin Deficiency with empty Fire rising with toothaches; Liver Yang rising or Liver Fire with dizziness, headache and blurred vision; damp-heat pouring downward with knee pain, or lower back painful obstruction; an auxiliary for painful urinary dysfunction (Lin) or vaginal discharge, especially for Stone Lin with lower back pain and bloody urine.

Huai Niu Xi's further main pharmacological effects include:
(i) Analgesic and anti-inflammatory: intravenous, intraperitoneal, subcutaneous or oral administration of Huai Niu Xi have marked analgesic and anti-inflammatory effects.
(ii) Cardiovascular: a mild hypotensive effect with a short duration of action.
(iii) Uterine stimulant: stimulating smooth muscle and leads to uterine contraction.

Huai Niu Xi tastes bitter and sour. Entering meridians: Liver, Kidney. Dosage: 6-16 g. Tincture: 2-4 ml.

Jiang Huang (Pharmacological Name: Rhizoma Curcumae Longae; Other names: Turmeric Rhizome, Bao Ding Xiang Curcumin, Indian saffron; Pin Yin Name: Jiang Huang): an east Indian plant, a member of the ginger family used as a spice and food color. Roots are well developed into a bundle. Underground stems are used in some cultures to treat certain stomach problems. The substance in Jiang Huang that gives it a yellow color (curcumin) is used for treatment of cancer, Alzheimer disease, cystic fibrosis, and psoriasis.

Jiang Huang affects Spleen and Liver meridians. Its main functions include breaking stasis to promote circulation of Qi and inducing menstruation to relieve menalgia; opening of channels and collaterals to alleviate pain; expelling Wind and invigorating the Blood; reducing swelling; relieving stagnation of Qi (e.g., Liver stagnation), hypochondriac pain, dysmenorrhea, amenorrhea, postpartum abdominal pain due to blood stasis from Deficiency Cold, wind-damp arthralgia, swelling and pain from sores, lesions and bruises. Recommended dosage is from 3 to 10 grams.

Jiang Huang's further main pharmacological effects include:
(i) Llowering both cholesterol and triglyceride levels.
(ii) Anti-inflammatory.
(iii) Antiplatelet: inhibits aggregation of platelets.
(iv) Cholagogic: many components of Liang Huang have marked effectiveness in increasing the production and excretion of bile.
(v) Uterine stimulant: administration of liang Huang decoction via intraperitoneal or subcutaneous routes can terminate pregnancy, presumably by stimulation and contraction of the uterus.
(vi) Cytotoxic and antitumor effects via modulating cell migration and cell-cell adhesion.
(vii) Anti-proliferative activity.
(viii) Immunostimulatory activities.

Curcumin (Jiang Huang) tastes bitter, acrid. Entering Meridians: Liver, Spleen, Stomach. Dosage: 3-10 g. Tincture: 1-4 ml.

Blood moderator herbs, which can substitute or be alternatives to Jiang Huang in contemplated herbal formulations, are the same as those that can substitute Yan Hu Sou.

### Internal Body Heat Cooling Herbs

The term “Internal Heat cooling herbs”, as used herein, refers to herbs which alleviate symptoms of excess Heat inside the body. This class of herbs is typically used in the treatment of diseases that are characterized by infection and inflammation. This class comprises 3 subcategories: “Fire Heat”, “Blood Heat”, and “Toxic Heat”.

The Fire and Heat cooling herbs are used to treat acute inflammatory or infectious diseases such as encephalitis (inflammation of the brain), pneumonia (inflammation of the lungs) and acute bronchitis (inflammation of the bronchial mucous membrane). Non-limiting examples include Shi Gao (Gypsum Fibrosum) and Xia Ku Cao (Spica Prunellae Vulgaris).

Herbs that reduce Blood Heat are generally used to stop bleeding and are helpful in conditions such as nose bleeds, vaginal bleeding or any other abnormal bleeding occurring in the body. Non-limiting examples include Zhi Mu (Rhizoma Anemarrhenae Asphodeloidis), Mu Dan Pi (Cortex Moutan Radicis) and Xuan Shen (Radix Scrophulariae Ningpoensis).

Herbs that reduce toxic heat are normally utilized for inflammatory and infectious diseases as well as many skin diseases, mumps, mastitis, lymphangitis, and appendicitis. Non-limiting examples include Huang Lian (Rhizoma Coptidis), Huang Qin (Radix Scutellariae Baicalensis), Huang Bai (Cortex Phellodendri), Zhi Zi (Fructus Gardeniae Jasminoidis) and Long Dan Cao (Radix Gentianae Longdancao).

Xia Ku Cao (Scientific name: Spica Prunellae; Common Names: Prunella, Selfheal Spike, Allheal Spike, Woundwort; Pin Yin Name: Xia Ku Cao). Xia Ku Cao is the spike or whole plant of the perennial herb, Prunella vulgaris L. (Family Labiaceae), mainly found in Chinese provinces. The fruit turns brownish red in the summer and is left to dry in the sun. Xia Ku Cao is light in weight, faint in odor and mild in taste. It is used for reducing inner Heat and detoxification. It can clear and purge Liver Heat, treat poor vision and infection. Xia Ku Cao is applied externally as healing herb in minor cuts, and mouth sores. It can reduce blood pressure and blood sugar level. It is applied to provide relief to women who have distress during menstruation, bloating or indigestion, urinary problems, or heavy bleeding during menstruation causing anemia.

Further actions attributed to Xia Ku Cao include clearing Liver Fire and brightening the eyes; clearing Hot Phlegm and dissipating nodules; and hypertension. Main indications/symptoms treatable with Xia Ku Cao include Liver Fire with red, painful or swollen eyes or headache and dizziness, double vision, watery eyes; eye pain which is worse in the evening due to Liver Yin Deficiency; neck lumps or nodules due to Phlegm-Fire such as scrofula, lipoma, carbuncle, swollen glands, goiter thyroid or enlarged lymph glands.

Xia Ku Cao's main pharmacologic effects:
(i) Antihypertensive effect by reducing blood pressure, attributed to vasodilation. The stems, leaves, flowers and entire plant all have antihypertensive functions.
(ii) Anti-inflammatory action, presumably via increased secretion of ACTH and/or glucocorticoids.
(iii) Antibiotic effects against Shigella spp., Salmonellatyphi, E. coli, Pseudomonas aeruginosa, Mycobacterium tuberculosis, Streptococcus spp., and dermatophytes.
(iv) Smooth muscle stimulant.
(v) Antiestrogenic activity: inhibition of estrogen signaling through depletion of estrogen receptor alpha. Xia Ku Gao extract, at non-toxic concentrations, significantly reduced alkaline phosphatase activity and cell proliferation in response to estrogen in a dose-dependent manner.
(vi) Antioxidant.

Xia Ku Gao tastes bitter acrid (slightly sweet). Entering meridians: Gallbladder, Liver, Lung, Kidney. Dosage: 6-18 g. Tincture: 2-5 ml.

Heat cooling herbs, which can substitute or be alternatives to Xia Ku Gao in contemplated herbal formulations include, but are not limited to, Cha Chi Huang (Herba Stellariae Aquaticae); Ren Zhong Bai (Urinae Hominis Sedimen); and Zhi Zi (Gardeniae Jasminoidis, Fructus).

Chai Hu (Pharmaceutical name: Radix Bupleuri; Common names: Bupleurum Root, Hare's Ear Root, Thorowax Root, Red Thorowax Root, Asian Buplever Root; Pin Yin Name: Chai Hu). Exterior cooler, prepared from the dried roots of Bupleurum Chinese DC for harmonizing and soothing GAN (Liver) Qi stagnation. Its active components are saikosaponins, one of which, saikosaponin-d, exhibits anticancer effects via autophagy induction. This is one of the most frequently used herbs in Chinese herbal medicine and has major functions depending on dosage and combination with other herbs. Main actions: resolves Shao Yang disorders and reduces fever (harmonizes the Exterior and Interior); spreads Liver Qi and relieves stagnation; raises Yang Qi (specifically the clear Qi of the stomach and gallbladder); dissipates Wind-Heat; and resolves phlegm and congestion. Main indication/symptoms treatable with Chai Hu include Shao Yan Stage with alternating chills and fever, a bitter taste, dizziness, tinnitus, flank pain, irritability, vomiting and a stifling sensation in the chest; Heat Deficiency (auxiliary); emotional instability and menstrual problems; disharmony between the Liver and Spleen with epigastric and flank pain, abdominal bloating, nausea and indigestion.

Chai Hu's further main pharmacological effects include:
(i) Analgesic and antipyretic: essential oil, saikosaponin, and decoction of Chai Hu all have marked analgesic and antipyretic effects.
(ii) Sedative: Chai Hu and saikosaponin extracted therefrom have a sedative effect.
(iii) Anti-inflammatory action by decreasing capillary permeability caused by histamine and 5-hydroxy-tryptamine.
(iv) Hepatoprotective.
(v) Cholagogic effect, achieved by increased production and excretion of bile.
(vi) Antihyperlipidemic: Saikosaponin A and D have marked effective in reducing triglycerides and moderate influence in reducing cholesterol.
(vii) Immunostimulant: stimulates both humoral and cellular immunity.
(viii) Antibiotic effect against beta-hemolytic streptococcus, Vibrio cholerae, Mycobacterium tuberculosis, leptospira, some influenza viruses, poliomyelitis viruses, and hepatitis viruses.

Chai Hu tastes bitter acrid (Astringent). Entering meridians: Gallbladder, Liver, Pericardium, San Jiao. Dosage: 3-12 g. Tincture: 1-3.5 ml.

Body Heat cooling herbs which can substitute or be alternatives to Chai Hu in herbal formulations disclosed herein include, but are not limited to, Bo He (Herba Menthae Haplocalycis); Man Ji Zhi (Viticis, Fructus); Mao ZI Dun Tou (Herba Abutilon Indicum); Sang Ye (Mori Albae, Folium); and Yi Zhi Xiang (Herba Vernoniae Cinereae).

Mu Dan Pi (Pharmaceutical name: Cortex Moutan; Common names: Tree Peony Root Bark, Moutan Root Bark; Pin Yin Name: Mu Dan Pi). Originates from the root bark of Paeonia suffruticosa Andr. of family Ranunculaceae. Collected in autumn all over China. Clears Heat and cools Blood, activates or invigorates Blood, resolves stasis, reduces Heat Deficiency, clears Deficiency Fire, clears Liver Fire.

Main indications/symptoms treatable with Mu Dan Pi include syndrome of Heat entering the Blood system in warm diseases, which is manifested as fever aggravated at night; macula or papule; hematemesis and apostaxis. It can be combined with other Blood-cooling, toxicity-removing and bleeding-stopping herbs to reinforce its action of clearing Blood Heat as well as preventing the side effects of Blood-Cooling and bleeding-stopping herbs. In syndrome of Blood stasis, it is wildly indicated for irregular menstruation, amenorrhoea, abdominal mass and traumatic injury caused by Blood stasis. For irregular menstruation accompanied with transformation of depressed Liver Qiinto Fire, it is often combined with menstruation-regulating herbs. Mu Dan Pi is effective in curing abscess, dissipating nodulation and stopping pain, and is commonly indicated for sores and abscess with swelling pain in body surface or internal organs caused by Fire-toxicity accumulation and Blood Heat stagnation.

Mu Dan Pi's further main pharmacological effects include:
(i) Anti-inflammatory actions, attributed to inhibition of prostaglandin synthesis and decreased permeability of the blood vessels.
(ii) Cardiovascular effects associated with increased blood perfusion to the coronary arteries, decreased cardiac output and decreased load on the left ventricle. Protective effect against ischemia of the heart.
(iii) Antihypertensive effect.
(iv) Antibiotic effect against Staphylococcus aureus, Beta-hemolytic streptococcus, Bacillus subtilis, E. coli, Shigelladysenteriae, Pseudomonas aeruginosa, Bacillus proteus, Diplococcus pneumoniae, and Vibrio cholerae.
(v) Others: sedative, antiseizure, antipyretic and immune-enhancing effects.

Mu Dan Pi tastes bitterly acrid. Entering meridians: Heart, Liver, Kidney. Dosage: 6-12 g; Maximum dose: 30 g. Tincture: 2-4 ml.

Heat cooling herbs, which can substitute or be alternatives to Mu Dan Pi in herbal formulations disclosed herein include, but are not limited to, Di Gu Pi (Cortex Lycii Radicis); Ji Guan Hua (Flos Celosiae Cristatae); Xi Jiao (Rhinoceri, Cornu); Xuan Shen (Scrophulariae Ningpoensis, Radix); Yin Chai Hu (Stellariae Dichotomae, Radix); and Zi Cao (Arnebiae Seu Lithospermi, Radix).

### Yang Tonic Herbs

Yang tonics are powerful herbs in Chinese herbalism and, like the Yin tonic herbs, affect primarily the “Kidney” function. Yang tonics generally have a “warm” or “hot” energy and are bestowed with the ability to build will power and courage. Yang energy is also associated with mental creativity and the ability to manifest one's ideas. Certain Yang tonic herbs are very famous as sexual tonics—some are even considered “aphrodisiac” since Yang energy is said to control sexual drive. Yang herbs are consumed for stimulating metabolism and building muscles. They support bone and are often consumed for supporting the skeleton, in particular the lower back, knees and other joints.

Xu Duan (Pharmaceutical name: Radix Dipsaci; Common names: Dipsacus Japanese Teasel Root Himalayan Teasel Root; Pin Yin Name: Xu Duan). Main actions of Xu Duan include tonifying the Liver and Kidneys, reconnecting and strengthening the sinews and bones, stopping uterine bleeding, calming the fetus and preventing miscarriage, promoting the movement of blood, alleviating pain, generating flesh, and reducing swellings, abscesses and sores.

Xu Duan is pharmacologically effective in nutritional deficiency and as antioxidant.

Xu Duan tastes bitter, sweet, acrid. Entering meridians: Liver, Kidney. Dosage: 6-12 g; Maximum dose: 30 g. Tincture: 1-4 ml.

Yang tonic herbs, which can substitute or be alternatives to Xu Duan in herbal formulations disclosed herein include, but are not limited to, Ba Ji Tian (Radix Morindae Officinalis); Bu Gu Zhi (Fructus Psoraleae Corylifoliae); Du Zhong (Cortex Eucommiae Ulmoidis); Gou Ji (Rhizoma Cibotti); Hai Gou Shen (Testes Et Penis Ostariae); Hai Tao Ren (Semen, Juglandis Regiae); Jiu Zi (Semen Allii Tuberosi); Rou Cong Rong (Cistanches Deserticolae, Herba); She Chuang Zi (Fructus Cnidii); Tu Si Zi (Cuscutae Chinensi, Semen); and Xian Mao (Curculiginis Orchioidis, Rhizoma).

### Bleeding Stoppers

Bleeding stoppers are herbs that stop bleeding, useful, e.g., for uterine bleeding, excess menses, bleeding from trauma, vomiting of blood, nose bleeds, and blood in the urine. They play a hemostatic role, and their effects are classified into four types: (1) removing Blood stasis; (2) cooling Blood (3) warming meridians; and (4) astringing.

Type 1: herbs for removing Blood stasis and stopping bleeding. Common herbs of this type include Cortex Mouton (Danpitan), Pollen Typhae (Puhuangtan), Selaginella (Juanbaitan) and Radix Rubiae (Qiancaotan), Lotus leaf and the like. Raw Pollen Typhae is mainly used for promoting blood circulation and stopping pain, while charred Pollen Typhae is mainly used for stopping bleeding. Raw lotus leaves are characterized by relieving summer-heat, clearing Heat and sending up the lucid Yang, while carbonized lotus leaves strong induce astringency, remove blood stasis and stop bleeding.

Type 2: herbs for cooling Blood and stopping bleeding. These herbs can restore over speed Blood circulation caused by Heat thus avoiding bleeding. Mainly applied to the treatment of bleeding symptoms caused by exuberant Fire and Heat, Fire excess from Yin Deficiency, superficial venules and lymph vessels and forced exuberant blood circulation. Common herbs for cooling Blood and stopping bleeding include Cirsium Japonicum DC (Dajitan), Cirsium Setosum MB (Xiaojitan), Sophora Japonica (Huaihuatan), Sanguisorba Root (Diyutan) and Radix Scutellariae (Huangqintan), and the like.

Type 3: herbs for warming meridians and stopping bleeding. They can be applied to Cold Deficiency hemorrhage syndromes caused by Spleen failing to manage Blood and unstable Chong Channel, such as hematochezia, uterine bleeding, purpura and dark tint face. Non-limiting examples include raw Artemisia Argyi, which is used for cooling Blood and stopping bleeding with Cold nature. After processing, charcoal Artemisia Argyi may be used for warming meridians and stopping bleeding with Warm nature and often applied to treatment of insufficiency-cold female bellyache, uterine bleeding, dysmenorrhea and postpartum abdominal pain.

Type 4: herbs for astringing and stopping bleeding. Applied to patients with endless massive bleeding, and are mainly used to treat functional uterine bleeding, induced abortion bleeding and other external causes of gynecological bleeding. Common herbs for astringing and stopping bleeding include palm (Zonglvtan), Nodus nelumbinis Rhizomatis (Oujietan).

Further bleeding stoppers include, e.g., Folium Artemisiae Argy, Radix Notoginseng, and Rhizoma Bletillae Striatae.

In one aspect, the present disclosure relates to herbal formulations comprising at least one herb, or a component thereof, from three or more different classes of Chinse medicinal herbs selected from Blood tonic, Blood moderators, Blood heat coolers, body heat coolers, bleeding stopper and Yang tonic herbs, as defined herein, in any combination thereof. For example, a disclosed formulation may comprise three, four, five, six, seven, eight, nine, ten, eleven, twelve, thirteen or more Chinese medicinal herbs, provided that the herbs in the formulation are from at least three different classes. In some embodiments, the herbs, in any combination thereof, are useful at least for treating an inflammatory mediated pathology of the pelvis and abdomen.

The term “formulation” refers to a material or mixture prepared according to a specific formula. In the context of the present disclosure, a formulation is a medicinal preparation also referred to as “medicinal herbal product”, “herbal formulation” or “medicinal herbal composition”, comprising three or more medicinal herbs or components thereof, from three or more different classes of Chinse medicinal herbs, prepared according to a particular formula. A contemplated formulation may be prepared for administration in a specific form and/or a specific route. A formulation is the basic herbal composition from which certain dosage forms are produced or fabricated.

The term “dosage form” refers to the physical characteristics of a drug product, which contains the active principal ingredients (APIs) and invariably other ingredients, such as excipients, fillers, flavours, preservatives, emulsifiers, etc. In other words, “dosage form” is the form in which a drug product is marketed for use. In the context of the present disclosure, a dosage form, also referred to as “herbal dosage form”, comprises a specific mixture of herbs, e.g., Chinese herbs, fragments and/or components thereof, and inactive components (excipients), in a particular configuration, and apportioned into a particular dose. In the context of some embodiments described herein, the term “dosage form” can also sometimes refer only to the pharmaceutical formulation of the medicinal herbals and/or active components thereof, without considering matters beyond that (like how it is ultimately configured as a consumable product such as a capsule, tablet, solution etc.). Thus, the term “dosage form” is sometimes interchangeable herein with the term “formulation”.

A “unit-dose”, as used herein, is an amount or unit of a compound, mixture, or preparation containing a controlled amount of substance that is separately identifiable and in a form that indicates that it is the amount or unit by which the substance is separately administered to or taken by an individual. Unit dose also means a sealed single-unit container designed such that the contents are administered to the patient as a single dose, directly from the container. Each unit dose container bears a separate label. A unit dose, in embodiments described herein, is a medicinal herbal product containing controlled amounts of multiple herbs (or specific components thereof), as defined herein. A contemplated herbal formulation may be fabricated and marketed for use as a single unit-dose dosage form, namely a product that contains a single unit-dose. A contemplated herbal formulation may alternatively be fabricated and marketed for use as a multiple unit-dose dosage form, namely a product that contains at least two unite-doses, which may have the same or different herbal constituents and/or the same or different physical characteristics, optionally packaged together.

Dosage forms may be in solid, liquid or semi-solid forms. Solid dosage forms include, for example, tablets, capsules, powder, and the like. Semi-solid dosage forms include, e.g., pasts, creams gels and the like. A liquid dosage form, e.g., of herbs and/or components thereof, includes, for example, solutions, emulsions or suspensions.

Dosage forms suitable for oral administration include, but are not limited to, pills, tablets (e.g., buccal, sublingual or chewable tablets), capsules, powders, pastilles, lozenges, granules, liquid solutions or suspensions (e.g., drinks, elixirs, syrups, oral drops), pastes, buccal films, and oils. Dosage forms suitable for topical administration include, but are not limited to, pastes, creams, gels, oils, ointments, lotions, dusting powders, skin patch, liniments, solutions and suspensions.

Various dosage forms may exist for a single, particular medicinal herbal product, since different medical conditions can warrant different routes of administration. For example, persistent nausea, especially with vomiting, may make it difficult to use an oral dosage form and instead, in such a case, it may be necessary to use an alternative route such as inhalational, buccal, sublingual, nasal, suppository or topical routs of administration. Additionally, a specific dosage form may be a requirement for certain kinds of herbs, as there may be issues with various factors like stability, bioavailability and/or pharmacokinetics.

In some embodiment, a disclosed formulation or a disclosed dosage form comprises 3 or more Chinese herbs, for example, 3, 4, 5, 6, 7, 10, 11, 13, 15 or more herbs that belong to at least three different categories or classes as defined herein.

The relative amount of a given Chinese herb or components thereof in a contemplated formulation may be in the range of from about 0.1% to about 50% by weight (w/w) or by volume (v/v) of total formulation. For example, from about 0.1% to about 10%, from about 0.1% to about 20%, from about 0.5% to about 10%, from about 0.5% to about 15%, from about 1% to about 10%, from about 1% to about 15%, from about 1% to about 20%, from about 5% to about 20%, from about 5% to about 40%, from about 10% to about 30%, from about 15% to about 45%, from about 0.1% to about 1%, from about 0.5% to about 2.5%, from about 0.5% to about 5%, from about 1.5% to about 2.5%, from about 2% to about 3.5%, from about 3% to about 6%, from about 4% to about 5.5%, from about 5% to about 8%, from about 6.5% to about 8%, from about 5% to about 15%, from about 8.5% to about 10%, from about 10% to about 15%, from about 12% to about 20%, from about 15% to about 25%, from about 20% to about 25%, from about 20% to about 30%, from about 25% to about 45%, from about 30% to about 40%, or from about 42% to about 50% (w/w) or (v/v), and any subranges and individual values therebetween.

In some embodiments, the herbs in the formulation are provided in their entirety, i.e., as whole and intact entities.

In some embodiments, the herbs are subjected to various manipulations such as extraction, and a contemplated formulation may comprise herbal extracts, e.g., oils, water-based or alcohol-based extracts and any combination thereof.

In some embodiments, specific active components are produced from the herbs, and a contemplated formulation may comprise only specific herbal components.

In some embodiments a contemplated formulation may comprise a combination of intact herbs, herbal extracts and/or specific herbal components.

The contemplated formulations disclosed herein may be fabricated into various dosage forms. For example, in some embodiments, the herbs are boiled to yield an extract, which is then concentrated to a paste. The paste can then be dried to a powder (preferably a fine powder). Optionally, powder forms of at least three herbs from at least three different classes/categories are mixed and encapsulated into a capsule suitable for ingestion.

In some embodiments, at least seven herbs from at least three different classes are combined to produce a contemplated formulation for treating an inflammatory mediated pathology of the pelvis and abdomen, for example, for treating endometriosis. In some embodiment, a contemplated formulation comprises a mixture of 7-13 herbs from three or more different categories. In some embodiments, these mixtures are in the form of powders.

In some embodiments, a disclosed formulation comprises curcumin extract and/or Jiang Huang. In Chinese medicine, Jiang Huang or parts of the plant (e.g., leaves, stem, flowers etc.) are defined and valued by their energetic qualities and not necessarily by the specific components they contain, e.g., curcumin.

In some embodiments, Jiang Huang or curcumin is a sole Blood moderating herb or component thereof, respectively, in the formulation.

In some embodiments, curcumin and/or Jiang Huang are additional herbal Blood moderating ingredient in the formulation.

An exemplary formulation comprising 7 medicinal Chinese herbs (herein designated Formulation (I)) is described in Table 1:

TABLE 1
Formulation (I)

| Herb Name: | Amount (w/w) |
|---|---|
| San Leng | 6.5%-8% |
| Dan Shen | 8.5%-10% |
| Bai Shao | 8.5%-10% |
| Mu Dan Pi | 8.5%-10% |
| Chai Hu | 4.0%-5.5% |
| Pu Huang | 2.0%-3.0% |
| Xu Duan | 6.0%-8.0% |

A further exemplary formulation comprising 13 medicinal Chinese herbs and Curcumin extract (herein designated Formulation (II)), useful for treating e.g., endometriosis is described in Table 2 (the herbs are listed in no order, and their pharmaceutical names are indicated):

TABLE 2
Formulation (II)

| Herb Name: | Amount (w/w) |
|---|---|
| Pu Huang (Typhae, Pollen) | 2%-5% |
| Yan Hu Suo (Rhizoma Corydalis Yanhusou) | 6%-9% |
| Hong Hua (Carthamus Tinctorius) | 2%-6% |
| San Leng (Rhizome Scirpus) | 6%-9% |
| Xia Ku Cao (Prunellae Vulgaris, Spica) | 6%-8% |
| Bai Shao (Radix Paeoniae Lactiflorae) | 8%-10% |
| Chai Hu (Radix Bupleuri) | 3%-9% |
| Dang Gui (Radix Angelica Sinensis) | 2%-10% |
| Dan Shen (Radix Salviae Miltiorrhizae) | 6%-14% |
| Mu Dan Pi (Mutan Radicis, Cortex) | 8%-12% |
| Xu Duan (Dipsaci Asperi, Radix) | 6%-12% |
| Huai Niu Xi (Radix, Achyranthis Bidentatae) | 6%-10% |
| Jiang Huang (Rhizoma, Curcuma Longa) | 3%-9% |
| Curcumin (95% purity) | 10%-20% |

In some embodiments, a contemplated formulation comprises the herbs and curcumin extract listed in Table 2, and further comprises Qing Dai (amount 12.7% w/w; herein Formulation (III)).

In some embodiments, a contemplated formulation comprises the herbs listed in Table 2, but the curcumin extract is replaced by Qing Dai (amount 12.7% w/w; herein Formulation (IV)).

The herb Qing Dai, also known as Indigo naturalis, belongs to the Chines herbal category “heat and toxicity purifiers”, which differs from body heat cooling and Blood cooling herbs. Qing Dai contains natural ingredients such as indigo, indirubin, isoindigotin, and nimbosterol. In China, Qing Dai has been traditionally used as an antipyretic, an antiphlogistic, and as a hemostatic remedy. Qing Dai enemas have been used for ulcerative proctitis and are currently described in Chinese medical guidelines for the treatment of UC. Part used: Leaf. Entering meridians: Liver, Lung and Stomach. Qing Dai clears heat, resolves fire toxicity, cools the Blood, reduces maculae, disperses stagnation, reduces swelling, drains Liver fire, dispels summer heat, extinguishes wind to stop tremors, and drains Lung heat.

Formulations comprising both Qing Dai and curcumin provide balanced heating or cooling effects.

Further exemplary formulations contemplated by the present disclosure, herein designated Formulations (V)-(VII), are described in Tables 3-5:

TABLE 3
Formulation (V)

| Herb Name: | Amount (w/w) |
|---|---|
| Pu Huang (Typhae, Pollen) | 6%-12% |
| Yi Mu Cao (Leonuri Heterophylli, Herba) | 6%-24% |
| Hong Hua (Carthamus Tinctorius) | 4%-8% |
| San Leng (Scirpus, Rhizome) | 6%-12% |
| E Zhu (Rhizoma Curcumae Ezhu) | 6%-12% |
| Bai Shao (Radix Paeoniae Lactiflorae) | 8%-12% |
| Dang Gui (Radix Angelica Sinensis) | 2%-10% |
| Dan Shen (Radix Salviae Miltiorrhizae) | 6%-10% |
| Mu Dan Pi (Mutan Radicis, Cortex) | 8%-18% |
| Xu Duan (Dipsaci Asperi, Radix) | 6%-12% |
| Ba Ji Tien (Radix Morindae Officinalis) | 6%-10% |
| Rou Cong Rong (Cistanches Deserticolae, HERBA) | 3%-9% |
| Qing Dai (Indigo Pulverata Levis) | 10%-30% |

TABLE 4
Formulation (VI)

| Herb Name: | Amount (w/w) |
|---|---|
| Yi Mu Cao (Leonuri Heterophylli, herba) | 6%-24% |
| San Leng (Scirpus, Rhizome) | 12%-30% |
| E Zhu (Rhizoma Curcumae Ezhu) | 12%-30% |
| Bai Shao (Radix Paeoniae Lactiflorae) | 8%-12% |
| Dang Gui (Radix Angelica Sinensis) | 6%-10% |
| Xu Duan (Dipsaci Asperi, Radix) | 9%-18% |
| Rou Cong Rong (Cistanches Deserticolae, Herba) | 9%-18% |
| Curcumin extract | 10%-30% |
| Qing Dai (Indigo Pulverata Levis) | 20%-30% |

TABLE 5
Formulation (VII)

| Herb Name: | Amount (w/w) |
|---|---|
| Yi Mu Cao (Leonuri Heterophylli, HERBA) | 15%-30% |
| San Leng (Scirpus, Rhizome) | 12%-30% |
| Bai Shao (Radix Paeoniae Lactiflorae) | 20%-30% |
| Rou Cong Rong (Cistanches Deserticolae, Herba) | 20%-30% |
| Qing Dai (Indigo Pulverata Levis) | 20%-30% |

Additional herbal formulations contemplated herein are equivalents of the formulations disclosed herein. An “equivalent formulation”, as used herein, is a formulation based, e.g., on a formulation disclosed herein, e.g., Formulation (I), (II), (IIa), (III), (IV), (V), (VI), (VII) or (VIII), or derived therefrom, in which one or more of the herbs and/or herbal ingredients are replaced by other herbs/herbal ingredients from the same or different categories, and the formulation exerts a similar or identical therapeutic effect.

Equivalent formulations, as referred to herein, further encompass formulations based on formulations disclosed herein, e.g., Formulation (I), (II), (IIa), (III), (IV), (V), (VI), (VII) or (VIII), and comprise additional one or more herbs and/or herbal ingredients from the same or different categories, provided that the formulations exert similar or identical therapeutic effects as those of Formulation (I)-(VIII).

In some embodiments, a herbal formulation comprising 3-20, for example, 4, 5, 6, 7, 8, 9, 10, 11, 12, 13, 15, 17, 18, 19, or 20 Chinese medicinal herbs is fabricated for oral administration as a solid or liquid dosage Form. In some embodiments, the solid dosage form is a capsule, pill, caplet, powder, tablet, and the like.

The Chinese herbal formulations disclosed herein are advantageous over the state-of-the-art formulations mainly because they contain herbs form additional herbal groups/classes, such as Blood coolers, Internal Heat coolers and more. These additional groups of herbs have benefits in contributing to the immediate signs and symptoms of the treated diseases as well as long term improvement and even remission.

The formulations disclosed herein provide desired delicately co-balanced effects. It is noted that each of the herbs utilized in embodiments of the present disclosure has a diverse and wide range of activities and pharmacologic effects. When these herbs are combined, it is expected that both desired (e.g., beneficial) and undesired (e.g., harmful) effects will be reflected during treatment of a certain pathologic condition is a certain subject. It is also expected that due to such combinations, a beneficial activity of one herb will be compromised, diluted or even nulled by a contradicting activity of other herbs in the formulation. It is further expected that due to such combinations a harmful, adverse activity/effect of one herb will be intensified by the presence of other herbs in the formulation. Thus, it is appreciated that obtaining a desired balanced, harmonized pharmacologic/therapeutic effect of a contemplated combination of Chinese herbs is unpredictable and certainly surprising.

In some embodiments, the ingredients (herbs or components thereof) in a contemplated formulation are combined in “synergistic amounts”. By this it is meant that the amounts of the herbs (or components thereof) in the formulation is more effective in achieving the desired purpose (e.g., treating endometriosis symptoms) than when provided separately or in another herbal composition. Synergy indicates that the ingredients cooperate with each other to achieve an enhanced or superior result than would be achieved if either were administered alone, i.e., the effect is not additive.

### Methods of Treatment

According to traditional Chinese medicine philosophy, inflammatory and pain related diseases can be triggered by either access of a specific element or quality, or by the deficiency of an opposite element/quality. The element or quality may be tangible, e.g., matter, or non-tangible, e.g., energy. For example, access of the element “Internal Heat” or deficiency of the element “proper cooling action”. An improper or altered balance of elements/qualities is specific and unique for each individual. The herbal formulations and treatment regimen of the present disclosure are adapted to each individual and adjusted to address its specific needs.

Accumulated clinical experience of the present inventors is reflected herein in the design and successful provision of a therapeutic methodology, which is broad enough to tackle any common and/or diversified manifestation of an inflammatory and/or pain related disease. Thus, the present disclosure provides methods of treatment, which utilize combinations of Chinese medicinal herbs having the higher most potency accomplished by the careful selection of herbs that do not present contradicting effects, and by focusing on relevant qualities of the elected herbs. These methods are most suitable for Western clinical treatment as well (as demonstrated in Example 3 herein).

The methods disclosed herein are tailored to meet the specific needs of a patient (i.e., they are patient-specific) because they are based on provision of herbal formulations designed to target a patient's specific conditions in the context of the patient's whole physiological state. Yet, these methods are useful for treating, preventing, alleviating and/or mitigating a broad scope of inflammatory and/or pain related diseases, disorders and other conditions, and any implications and/or manifestations thereof.

In one aspect, the present disclosure relates to methods for treating, preventing, alleviating and/or mitigating pain. These methods comprise administrating to a subject in need thereof one or more of the Chinese herbal formulations disclosed herein.

According to TCM, pain occurs only if there is stagnation. In other words, pain is equal to stagnation: “where there is pain, there is stagnation, and where there is stagnation there is pain”.

Pain may be caused due to several reasons, one of which is Blood stagnation, one of the most important diagnostic conditions in Chinese medicine, because it is frequently the cause of intractable pain syndromes anywhere in the body, and particularly pain that is located or fixed at the lower abdominal area.

In some embodiments, the present disclosure relates to a method for treating, preventing and/or alleviating Blood stagnation, comprising administering to a subject in need thereof therapeutically effective amounts of one or more Chinese herbal formulations disclosed and/or suggested herein.

“A subject in need thereof”, in the context of these embodiments, is a subject suffering pain and/or diagnosed (e.g., by healer practicing CM) as afflicted with, or presenting one or more of the signs or symptoms of Blood stagnation as known in the art of CM, some of which are described herein.

Blood stagnation, also referred to herein as “Blood stasis”, is a condition of toxicity, of which the main distinguishing symptom is pain that is fixed in one place and is of a boring or stabbing character. This condition or situation is manifested by heavily restricted blood flow in all or parts of the body. When Blood stasis evolves, there is a tendency to form blood cloths, gathering of tissue, lumps and fibrosis such as in endometriosis.

Apart from a fixed stabbing pain, Blood stagnation is often diagnosed by signs on the skin such as purple blotches or bruises, purple lips, face and nails.

The organ that is most frequently affected by Blood stagnation is the Liver. Other organs that are commonly affected are the Heart, Lungs, Stomach, Intestines and Uterus.

In developing their therapeutic approach, the present investors addressed Blood stasis (stagnation) by developing a therapeutic model which focuses on the two main etiologies associated, directly or indirectly, with Blood stasis: Blood Deficiency (Xue Xu) and Yang Deficiency (Yang Xu).

Blood Deficiency is related to the quality and quantity aspects of the blood (Xue): it is a condition of “poor quantity”, that is followed by poor blood flow into the tissues, resulting in malnourished tissue, lack of proper immunity (an important factor in endometriosis) and eventually Blood stagnation. Blood Deficiency manifests in symptoms such as hair loss, dryness of tissues (especially mucus), weakness and improper function of the YANG MING system (consisting of the Stomach and the Colon).

Yang Deficiency, especially deficiency of the Kidney related Yang, is the cause of poor energy flow and movement, and inability to warm up the body (cold sensation). This is a deficiency of the quality that boosts, feeds or catalyzes physiological processes such as metabolic and hormone-mediates processes. Deficiency of Yang worsen stagnation in general, and Blood stasis in particular.

Other causes for Blood stagnation include:
Heat in the Blood: this may cause the blood to condense and therefore stagnate;
Interior Cold: cold congeals Blood which slows down its flow;
Phlegm, an aggravating factor that indirectly induces Blood stagnation;
Liver disorders: the Liver stores the Blood;
Spleen disorders: spleen maintains blood in the vessels;
Traumas or severe injuries; and
Some gynecological diseases.

It is noted that both Yang Xu and Xue Xu are the long-term etiologies which result in stasis, and embodiments disclosed herein focus on both.

A disclosed method for treating Blood stagnation provides immediate relief of symptoms and signs by, first, contradicting the Blood stasis via Blood moderators and Blood coolers (for example, Huang Teng (Caulis Fibraurea), Ji Guan Hua (Flos Celosiae Cristatae), Ji Xue Cao (Herba Centellae), Sheng Di Huang (Rehmanniae Glutinosae, Radix), Yin Chai Hu (Stellariae Dichotomae, Radix) and more), which are provided along with Internal Heat/Fire Coolers. Then, for a long-term effect, Blood tonic herbs along with Yang tonic herbs are provided, in order to ‘re-center’ the deviating system, promote remission an/or prevent progression of the disease.

Furthermore, a disclosed method for treating Blood stasis broadens the state-of-the-art therapeutic concept: it addresses both Blood Deficiency and Yang Deficiency. Therefore, the disclosed method is applicable and suitable not only for treatment of Blood stagnation but also for treatment or prevention of other diseases, disorders and conditions having Blood Deficiency and/or Yang Deficiency etiologies such as Internal Heat or Internal Cold.

State-of-the-art “classic” Chinese herbal formulations are designed based on specific attributes of the herbal ingredients: temperature, affected meridians and taste. As such, the intended uses of these formulations are based on the effect of each herb on the designated CM syndrome and meridian. Chinese herbal formulations that address the two deficiencies (Blood and Yang) which promote Blood stasis in gynecological context, were not known before the presently disclosed formulations were designed and developed.

Blood strengthening herbs are critically important in preserving a subject's essence and preventing its undue exploitation. Insufficient supply of Blood strengthening nutrients might lead to Blood Deficiency. In such circumstances, treatment with Blood strengthening herbs should precede application of Blood moderators so as avoid intensive flow of “empty Blood”. In TCM, the terms “essence” and “core aspect” are interchangeable and refer to a basic “material” which is stored in the body. A subject starts its life charged with a certain amount of this essence and exploits it throughout its lifetime. The core aspect determines the subject's ability to produce sperms/ovum, bone marrow and the like. Poor nutrition and/or extensive consumption/exploitation of resources might have adverse effects on the essence (e.g., extinction or emptying of the core aspect).

In some embodiments, the present disclosure relates to a method for treating, preventing and/or alleviating inflammatory and hormone mediated diseases, disorders and conditions, comprising administrating to a subject in need thereof, therapeutically effective amounts of one or more Chinese herbal formulations disclosed and/or suggested herein.

“A subject in need thereof”, in the context of these embodiments, is a subject suffering pain and/or diagnosed (e.g., by healer practicing CM) as afflicted with, or presenting one or more of the signs or symptoms of inflammatory and/or hormone mediated disease or condition as known to in the art of CM.

In some embodiments, a contemplated method is useful in treating, preventing and/or alleviating inflammatory and/or hormone mediated diseases, disorders and conditions of the pelvis and lower abdomen, including, but are not limited to, endometriosis, adenomyosis, chronic pelvic pain, painful bladder syndrome, interstitial cystitis, inflammatory bowel disease, irritable bowel syndrome, vulvodynia, vestibulodinia (vestiblitis), myofascial syndrome and associated morbidity including chronic fatigue syndrome, premenstrual syndrome (PMS) symptoms, mood changes and catamenia hemothorax.

In some embodiments, a contemplated method is useful for treating endometriosis.

Endometriosis is a gynecological condition in which bits of tissue lining the uterus (endometrium) grow in other parts of the body. Like in the uterus, this tissue builds up and sheds blood in response to monthly hormonal cycles. However, there is no natural outlet for the blood discarded from these implants, and it flows onto surrounding organs, causing swelling and inflammation. This repeated irritation leads to the development of scar tissue and adhesions in and/or around the endometrial implants.

Endometriosis most commonly strikes between the ages of 25 and 40. It can also appear in the teen years, but rarely before the start of menstruation. It affects the health and wellbeing of 6-10% of women of reproductive age and diagnosed in 50-60% of women with pelvic pain and up to 50% of women with infertility problems. Endometrial implants are most often found on the pelvic organs: the ovaries, uterus, fallopian tubes, and in the cavity behind the uterus. Occasionally, this tissue grows in distant parts of the body such as the lungs, arms, and kidneys. Newly formed implants, referred to as “peritoneal lesions”, may appear as small protuberances on the surfaces of organs and supporting ligaments. They may appear as white, red, vesicular or black “powder burn” lesions. Deep lesions are endometrial fibrotic lesions that penetrate deeper than 5 mm into the different organs and ligaments. Ovarian cysts (endometriomas) are cysts surrounded by endometrial tissue within the ovary and may range from pea to grapefruit size. Endometriosis is a progressive condition that usually advances slowly, over the course of many years. Cases are ranked from minimal to severe based on factors such as the number and size of the endometrial implants, their appearance and location, and the extent of the scar tissue and adhesions in the vicinity of the growths.

Although the exact cause of endometriosis is unknown, some factors are thought to influence the development of endometriosis, for example, heredity and immune system function. Women with endometriosis may have lower functioning immune systems that fails to eliminate stray endometrial cells. Exposure to dioxin (TCCD), a toxic chemical found in weed killers, is also believed to be linked to endometriosis.

While many women with endometriosis suffer debilitating symptoms, others have the disease without knowing it. Paradoxically, there does not seem to be any relation between the severity of the symptoms and the extent of the disease. The major symptoms are pelvic pain, dysmenorrhea, dyspareunia, infertility, gastrointestinal irregularities, urinary alterations, abnormal menstrual bleeding, i.e., heavy menstrual periods, irregular bleeding, and spotting.

Menstrual pain, a pain in the lower abdomen that begins a day or two before the menstrual period starts and continues through to the end, is typical of endometriosis. Some women also report lower back aches and pain during urination and bowel movement, especially during their periods. Pressure on the vagina and cervix causes severe pain for some women afflicted with endometriosis.

Other symptoms of endometriosis include chronic fatigue, nausea and vomiting, headaches, low-grade fevers, heavy and/or irregular periods, and hypoglycemia. Complications of endometriosis include internal scarring, adhesions, pelvic cysts, ruptured cysts, and bowel, bladder and ureteral involvement.

There is a strong association between endometriosis and infertility, although the reasons for this have not been fully explained. It is thought that the buildup of scar tissue and adhesions blocks the fallopian tubes and prevents the ovaries from releasing eggs. Endometriosis may also affect fertility by causing hormonal irregularities and a higher rate of early miscarriage.

How endometriosis is treated in Western medicine depends on the woman's symptoms, her age, the extent of the disease, and her personal preferences. The condition cannot be fully eradicated without surgery. Conservative treatment focuses on managing the pain, preserving fertility, and delaying progress of the condition.

Medical treatment is based on hormonal suppression of the endometriosis lesions/implants by combined oral contraceptives, progesterone only treatment and/or provision of gonadotropin releasing hormone (GnRH) agonists/antagonists. Medical treatment can be useful in managing pain symptoms but is incompatible with a desire for fertility. Discontinuation of treatment may lead to a relapse of symptoms and may cause patients to delay their fertility out of fear of recurring symptoms. In addition, some patients are not able to maintain symptom resolution or develop side effects, which discourage them from hormonal treatment. Patients with a family history of breast or ovarian cancer are concerned with the use of hormonal remedies.

Endometrial implants and ovarian cysts can be treated with laparoscopic surgery. Surgery is performed to restore anatomy, alleviate symptoms and improve fertility. Removing the uterus, ovaries, and fallopian tubes is the only permanent method of eliminating endometriosis. This is an extreme measure that deprives a woman at fertility age of her ability to bear children and forces her body into menopause.

In treating, alleviating and/or preventing at least some symptoms of endometriosis, disclosed medicinal Chinese herbal formulations can be given to endometriosis patients combined with hormonal therapy or as a substitute thereof. For example, patients that continuously suffer from symptoms of endometriosis despite medical therapy, or patients that need to discontinue hormonal treatment, may benefit from treatment with a disclosed herbal formulation.

In some embodiments, a disclosed method for treatment of endometriosis is combined with hormonal therapy.

The terms “therapy”, “treatment”, “treating”, “treat” as used herein are interchangeable and refer to: (a) preventing a disease, disorder, or condition from occurring in a human which may be predisposed to the disease, disorder and/or condition but has not yet been diagnosed as having it; (b) inhibiting the disease, disorder, or condition, i.e., arresting its development; (c) relieving, alleviating or ameliorating the disease, disorder, or condition, i.e., causing regression of the disease disorder and/or condition; and (d) curing the disease, disorder, or condition. In other words, the terms “therapy”, “treat,” “treatment,” and “treating,” extend to prophylaxis, namely, “prevent”, “prevention”, and “preventing”, as well as treatment per se of established conditions. Accordingly, use of the terms “prevent”, “prevention”, and “preventing”, would be an administration of the active agent to a person who has in the past suffered from the aforementioned conditions such as, for example, inflammation of the lower abdomen or endometriosis, but is not suffering from the conditions at the moment of the composition's administration.

Thus, the terms “treatment”, “therapy” and the like include, but are not limited to, changes in the recipient's status. The changes can be either subjective or objective and can relate to features such as symptoms or signs of the disease, disorder or condition being treated. For example, if the patient notes improvement in the ability to exert daily functions or decreased pain or pressure in the lower abdomen, then successful treatment has occurred. Similarly, if the clinician notes objective changes, such as by gynecologic examination, body temperature measurements, measuring the pulses in the wrists, looking at the tongue, examining the lower eyelid or gums, then treatment has also been successful. Alternatively, the clinician may note a decrease in the size of lesions (e.g., endometriosis implant) or other abnormalities upon examination of the patient. This would also represent an improvement or a successful treatment. Preventing the deterioration of a recipient's status is also included by the term. Therapeutic benefit includes any of a few subjective or objective factors indicating a desirable response of the condition being treated as discussed herein.

The term “therapeutically effective amount” as used herein, means the amount or dose of a compound, e.g., a combination of Chinses herbs, that when administered to a subject for treating a disease, disorder or condition as defined herein, is sufficient to effect such treatment for the disease, disorder or condition. The therapeutically effective amount may sometimes be the lowest dose level that yields a therapeutic benefit to patients on average, or to a given percentage of patients. The ‘therapeutically effective amount’ can vary depending on the herbal combination, the disease and its severity, and the age, weight, etc., of the subject to be treated.

Some embodiments of the present disclosure relate to oral administration, in a contemplated method, of one or more of Formulation (I), Formulation (II), Formulation (IIa), Formulation (III), Formulation (IV), Formulation (V), Formulation (VI), Formulation (VII), Formulation (VIII) and/or equivalents thereof, as defined herein. For example, one or more formulations equivalent to Formulations (I)-(VIII) may be administered to a subject in need thereof.

In some embodiments, a contemplated method comprises administration of one or more of Formulations (I)-(VIII) and/or equivalents thereof, supplemented with additional herbs of the same or different categories, curcumin extract and/or Jiang Huang.

In embodiments directed to treatment of endometriosis, at least one of Formula (I), Formula (II), Formula (IIa), or Formulae (III)-(VIII), and/or an equivalent thereof are provided in a liquid and/or solid dosage form for oral administration/consume. The solid dosage form may be, for example, tablets or capsules.

In some embodiments, the endometriosis patient is orally treated with Formulation (1) and curcumin extract. In some embodiments, the endometriosis patient is orally treated with Formulation (IIa) and/or equivalent thereof.

The daily amount (i.e., dose) of a Chinese herbal ingredient provided to a patient is calculated either based on (i) the herb's relative weight in a dosage form (% wight or w/w) and the total daily dosage (which, in turn, derives from the patient's wight); or (ii) the herb's relative weight in a dosage form, the total weight of the dosage form, and the number of dosage forms to be administered per day (which, in turn, is determined by the subject's body weight). For example, the daily amount (dose) in grams of herbal ingredient A is calculated as:

(Herb A×subject's weight (Kg))/10 grams

wherein “Herb A” is: (A's relative amount (%) in the dosage form)×(number of daily dosage forms). For Example, Bai Shao weight in daily dose for a 70 Kg patient is (9%×70/10)=0.63 grams.

An average daily dose of a disclosed herbal dosage form depends on the subject's need, clinical situation or circumstances, physical (e.g., age, weight) and/or physiological conditions. For example, an average daily dose of herbal capsules is about 8 to about 16 capsules (total weight of 4 grams to 8 grams), preferably 1 gram for every 10 kg of body mass. In some embodiments, such dose is taken one to about three times a day, for example, three times a day, for a period of 10-16 weeks.

In some embodiments, a dose of 1 gram for every 10 kg of body mass is administered, e.g., orally, three times a day, for a period of about 12 to 14 weeks.

The terms “comprises”, “comprising”, “includes”, “including”, “having” and their conjugates mean “including but not limited to”.

The term “consisting of” means “including and limited to”. As used herein, the singular forms “a” “an” and “the” include plural references unless the context clearly dictates otherwise. For example, the term “a compound” or “at least one compound” may include a plurality of compounds, including mixtures thereof.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Various embodiments and aspects as delineated hereinabove and as claimed in the claims section below find experimental support in the following examples.

### EXAMPLES

### Materials and Methods

Herbal formulations disclosed herein were prepared in various forms using procedures known in the art that preserved the desired activities of the herbs, such as, but not limited to, aqueous extracts, organic solvent (e.g., alcohol) extracts, and dry powder.

In some embodiments, the herbs were extracted, concentrated and dry powdered. The resulting powder was then processed/filled into pill/capsule shell. In line with the common practice in the herbal industry, 5:1 ratio powder was produced for each herb, namely each 1 gr of powder was produced from 5 gr of herb.

Herbs used in embodiments of the present disclosure were harvested from many locations, any time of the year. Herbal formulations were prepared from herbs or parts thereof obtained in their natural form. Each one of the herbs was handled individually to obtain an effective fine powder. In some embodiments, each herb was cleaned and soaked for 1 hour at room temperature in an extraction pot comprising water (about 10 times the amount of herb), and then boiled at 100 °C for 1 hour. The resulting liquid was concentrated to 40-70%, and the concentrated liquid was dried. The resulting paste was spray dried with potato starch to obtain an effective powder. The herbs comprise active components at concentrations sufficient to treat a given indication.

### Example 1

### Preparation of Formulation (VIII)

Thirteen herbs in their powder form were prepared as described above in Materials and Methods. Desired amounts of each herb were mixed to obtain Formulation (VIII) as indicated in Table 6 below. Formulation II was filled into capsules.

TABLE 6
Main ingredients of Formulation (VIII)

| Herb Name | Relative amount (w/w) |
|---|---|
| Pu Huang (Typhae, Pollen) | 2.4% |
| Yan Hu Suo (corydalis yanhusou, rhizoma) | 9.5% |
| Hong Hua (Carthamus Tinctorius) | 4.8% |
| San Leng (Rhizome Scirpus) | 7.1% |
| Xia Ku Cao (Prunellae Vulgaris, Spica) | 9.5% |
| Bai Shao (Radix Paeoniae Lactiflorae) | 9.5% |
| Chai Hu (Radix Bupleuri) | 4.8% |
| Dang Gui (Radix Angelica Sinensis) | 9.5% |
| Dan Shen (Radix Salviae Miltiorrhizae) | 9.5% |
| Mu Dan Pi (Mutan Radicis, Cortex) | 9.5% |
| XuDuan (Dipsaci Asperi, Radix) | 7.1% |
| Huai Niu Xi (Radix, Achyranthis Bidentatae) | 9.5% |
| Jiang Huang (Rhizoma, Curcuma Longa) | 7.1% |

### Example 2

### Preparation of Formulation (IIa)

Fourteen herbs in their powder form were prepared, and desired amounts of each herb were mixed to obtain Formulation III as indicated in Table 7 below. Formulation (IIb) was filled into capsules.

TABLE 7
Main ingredients of Formulation (IIa)

| Herb Name | Relative amount (w/w) |
|---|---|
| Pu Huang (Typhae, Pollen) | 2.2% |
| Yan Hu Suo (Corydalis Yanhusou, Rhizoma) | 8.3% |
| Hong Hua (Carthamus Tinctorius) | 4.2% |
| San Leng (Scirpus, Rhizome) | 6.2% |
| Xia Ku Cao (Prunellae Vulgaris, Spica) | 8.3% |
| Bai Shao (Radix Paeoniae Lactiflorae) | 8.3% |
| Chai Hu (Radix Bupleuri) | 4.2% |
| Dang Gui (Radix Angelica Sinensis) | 8.3% |
| Dan Shen (Radix Salviae Miltiorrhizae) | 8.3% |
| Mu Dan Pi (Mutan Radicis, Cortex) | 8.3% |
| Xu Duan (Dipsaci Asperi, Radix) | 6.2% |
| Niu Xi (HUI) | 8.3% |
| Jiang Huang (Rhizoma, Curcuma Longa) | 6.2% |
| Curcumin (95% purity) | 12.07% |

### Example 3

### Clinical Trial: Treatment of Endometriosis with Herbal Formulations

The effect of a contemplated herbal formulation on females afflicted with endometriotic pain and other symptoms was assessed. The primary endpoint was improvement in pain measures among women suffering from endometriosis symptoms, following consumption of the herbal formulation. The secondary endpoints were improvement in life quality, reduction in consumption of pain relievers and reduction of missing workdays.

The study was a double blinded randomized placebo-controlled trial. Fifty-four patients were enrolled in the study and control groups.

Women (patients) recruited for the study, either to the test group or the control group, were selected based on the following main inclusion criteria:
(a) pain symptoms reported by the patient, with or without analgetic treatment;
(b) age: 18-45 years old;
(c) the patient did not undergo surgical operation for partial or complete removal of abdominal organs, except for endometriosis lesions, ovarian cysts and fallopian cysts;
(d) the patient was not allergic to any of the ingredients in the herbal formulation provided during treatment;
(e) the patient was willing to use contraceptives during the study (provided she was hormonally treated);
(f) the patient did not change, discontinued or began a hormonal therapy during 3 months prior to the study; and
(g) the patient did not change, discontinued or begin alternative medical treatment during a month prior to the study.

The main exclusion criteria were:
(a) patients that were unable to cease medical treatment for the entire period of the study;
(b) patient afflicted with one or more of: renal disorder, hepatic disorder, severe cardio-vascular disorder, chronic pancreatitis, diabetes mellitus, or gallstone;
(c) patients with abnormal laboratory test results indicating anemia, leucopenia, thrombocytopenia or coagulation disorders;
(d) patients with sepsis or a non-endometriosis related inflammation, and patients with another known cause of pain;
(e) pregnant or breastfeeding patient;
(f) patients that presented allergic reaction to at least one component of the herbal formulation used in the study; and
(g) patient treated with statins or anti-coagulants.

Patients with surgically or clinically diagnosed endometriosis who complied with the inclusion criteria and gave their consent to participate in the study, were randomly assigned in a ratio of 1:1 to the study and control groups. Patients in the treatment group received capsules containing Formulation (IIa) prepared as described in Example 2 above. Patients in the control group received placebo capsules. Treatment lasted 4 months. The study was approved by the Institutional Review Board.

The patients visited the Endometriosis Center (Lin Medical Center, Israel) three times at 2-month intervals. Three times during the study, namely, on initiation, after 2 months (midterm visit) and 4 months after initiation (final visit), the patients were physically examined and were requested to fill 2 questionnaires. Physical examination included gynecological examination and ultrasound. A blood test for blood count, chemistry, tumor markers and pregnancy test were performed.

The first questionnaire filled out by the patient was a clinical questionnaire, for recording clinical and demographic data. The purposes of this questionnaire were routine follow-up and obtainment of basic information about each patient. The information extracted from this questionnaire is summarized in Table 8.

The second questionnaire was Endometriosis Health Profile Questionnaire (EHP-30)?, also referred to herein as quality of life (QOL) questionnaire. The EHP-30 served for assessing subjective improvement in certain parameters related to the patients' quality of life during the study, and for extracting quantitative results to confirm establishment of the secondary endpoints of the study such as reduction in consumption of pain relievers and reduction of missing workdays.

The core of the EHP-30 questionnaire consisted of 30 items (i.e., questions), divided into 9 categories: physical status, emotional status, work status, kids status, intercourse status, digestive status, health, physical pain, and last 4 week status. For each question, there were five optional answers available: “never”, “seldom”, “occasionally”, “very often” and “always”, which were scored 0, 1, 2, 3, and 4, respectively. The questions covered various aspects of daily life quality which were affected by endometriosis symptoms, e.g., emotional and physical well-being, behavior patterns, social activity, self-image, ability to work, relationship with family members, sexual intercourse, and the like. A comparison of life quality measures between the study and placebo groups was made for three time points of the study: entry, midterm and end. Changes in EHP questionnaire overtime (entry/midterm/end) in each group, separately, were assessed using the Friedman test followed by Wilcoxon sign rank test for pairwise comparisons with Bonferroni correction. The scores (answers) for a particular category (namely, for all questions in that category) in the questionnaire were averaged for all patients in each group and presented as EHP score versus each time point of the study.

Upon inclusion (entry) and midterm meetings, each patient received a 2-month dosage (altogether 4-months dosage) of herbal formulation. The herbal formulation was taken by the patients orally 2 times a day, for the entire period of the clinical study. The formulation was consumed separately from food, preferably, ½ hour or more prior to food consumption, or an hour after a meal. The formulation was prescribed per patient according to the following criterion: 1 g (of a 5:1 ratio powder) for every 10 Kg of body mass, which is an average of 5-7 grams per day. Approximately 6-9 capsules (800 mg each) per day per patient.

To evaluate treatment effect, patients filled out an electronic daily diary, which included recitation of pain intensity, menstruation parameters, usage of analgesics and loss of workdays. The information extracted from this daily follow-up was used for proving the primary endpoint of the study, namely, improvement in pain measures. For showing improvement in pain statistically, pain free days (PFD) were used as the clinical improvement measure, namely, how many days in a month were reported by patients in each group as pain free days. The percentage of pain free days during the month was calculated based on scores of a visual analogue scale (VAS) ranging from 0 to 10 cm. A VAS is one of the pain rating scales often used to measure the intensity or frequency of various symptoms such as the amount of pain that a patient feels, which ranges across a continuum from none to an extreme amount of pain. Scores are recorded by making a mark on a 10-cm line that represents a continuum between “no pain” and “worst pain.”

For weighted pain analysis, the total amount of pain per month was established using a weighted calculation based on the formula:

(mean pain per day×days with pain)/reported days

Follow-up calls for possible side effect assessment were made between the visits and one month after the end of the study.

The generalized linear model (GLM) repeated measure is a statistical technique that takes a dependent, or criterion variable, measured as correlated, non-independent data. It is Commonly used when measuring the effect of a treatment at different time points. GLM repeated measure was used for analyzing variance when the same measurement, namely, PFD or weighted pain analysis was made. This was done 4 times (4 months) for each subject. For analyzing the between-subjects factors, the two groups were specified (i.e., study and control groups). Using this general linear model procedure, null hypotheses were tested for both the between and within subject factors. The interactions between factors as well as the effects of individual factors were investigated, with emphasis on the interactions between factors.

Out of 54 patients, 41 completed the study: 22 in the study group and 19 in the control group. The main dropout reasons were difficulty in consuming the capsules, low or non-compliance with study requirements and wanting to start hormonal treatment.

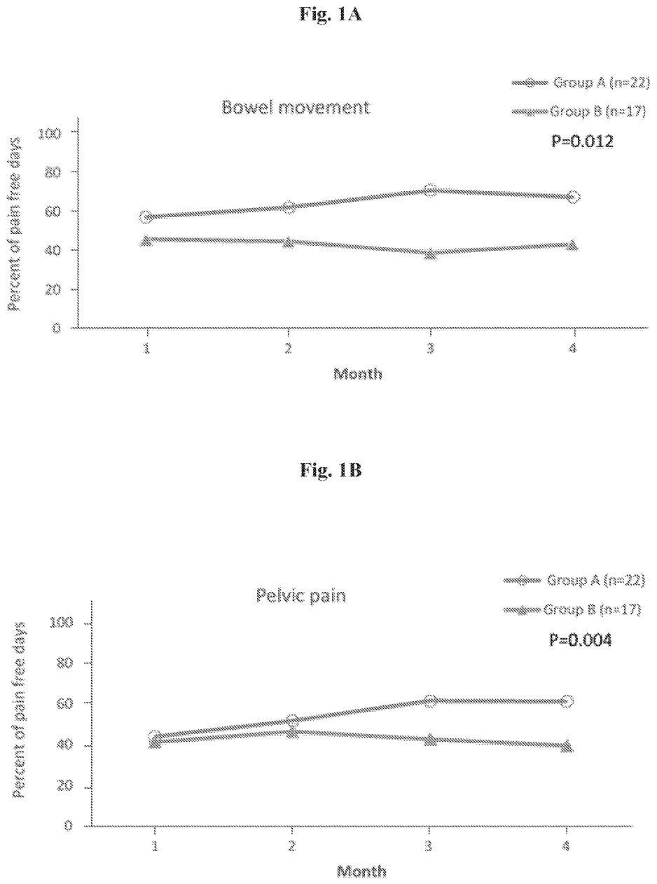

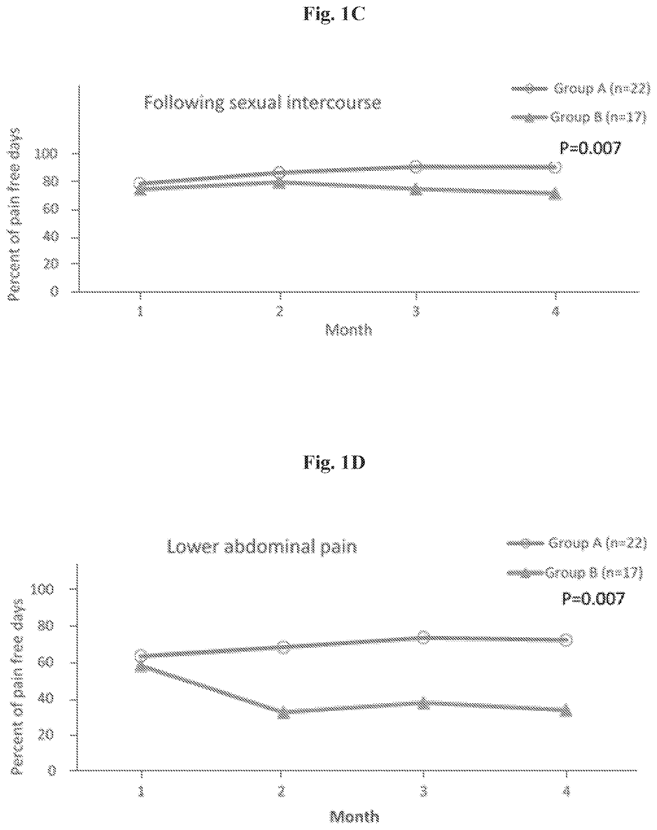

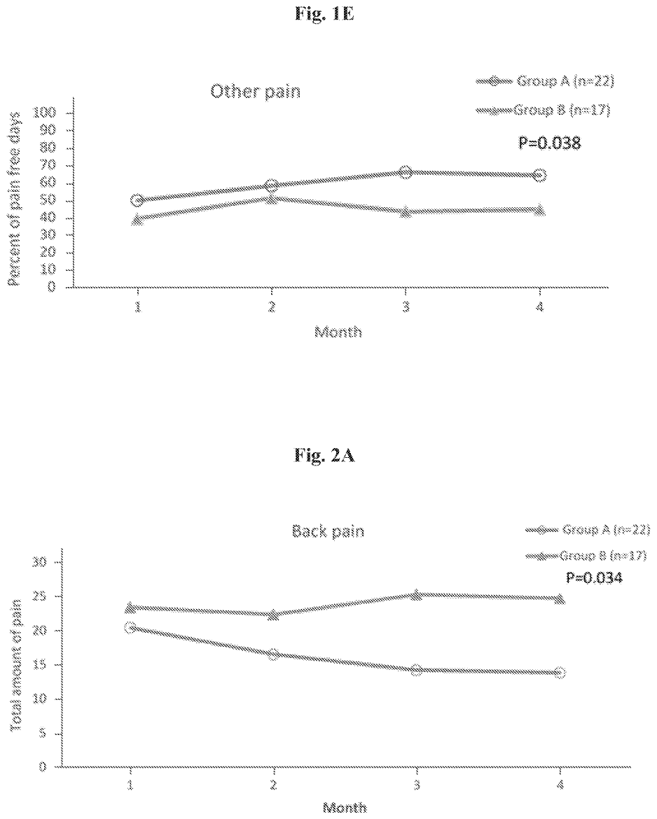

Final analysis of statistically significant improvement at the end of the 4-month treatment as compared to the symptoms before treatment for the study group for vs. placebo group was performed. The results are shown in FIGS. 1-3.

Patient demographics and baseline characteristics were similar among the groups (Table 8).

TABLE 8
Baseline characteristics and patient demographics (n = 41)

| | Study | Control | |
|---|---|---|---|
| | n = 22 | n = 19 | P |
| Age years; mean (*SEM) | 34.8 ± 5.4 | 33.2 ± 6.6 | 0.379 |
| Family status; n (%) | | | 0.96 |
| Single | 8 (36.4) | 7 (36.8) | |
| Married | 6 (27.3) | 4 (21.1) | |
| Partner | 7 (31.8) | 7 (36.8) | |
| Divorced | 1 (4.5) | 1 (5.3) | |
| Age at menarche; mean | n = 21 | n = 18 | 0.462 |
| (SEM) | 13.1 ± 1.03 | 13.4 ± 1.4 | |
| # periods last year; | n = 20 | n = 14 | 0.341 |
| mean (MIN, MAX) | 11.5 (9.3; 12) | 4.5 (0; 12) | |
| Regular cycles; n (%) | 15 (75.0) | 10 (71.4) | 0.817 |
| Duration of bleeding; | n = 18 | n = 14 | 0.512 |
| mean (SEM) | 5.5 ± 1.8 | 6.3 ± 2.4 | |
| Age of symptom onset; | n = 21 | n = 17 | 0.663 |
| mean (SEM) | 15.5 ± 5.1 | 15.0 ± 3.4 | |

*SEM, standard error of the mean

Analysis of the electronic daily diary showed similar baseline pain levels in both groups followed by a significant increase in pain-free days (PFD) observed in the study group compared to the control group, for most of the pain parameters (FIGS. 1A-1E). Significant differences were found with regards to lower abdominal (p=0.007) and pelvic pain (p=0.004) as well as pain during bowel movements (p=0.012), during sexual intercourse (p=0.007) and other types of pain (p=0.038). No significant differences were found with regards to back pain, rectal pain, thigh pain or pain during urination.

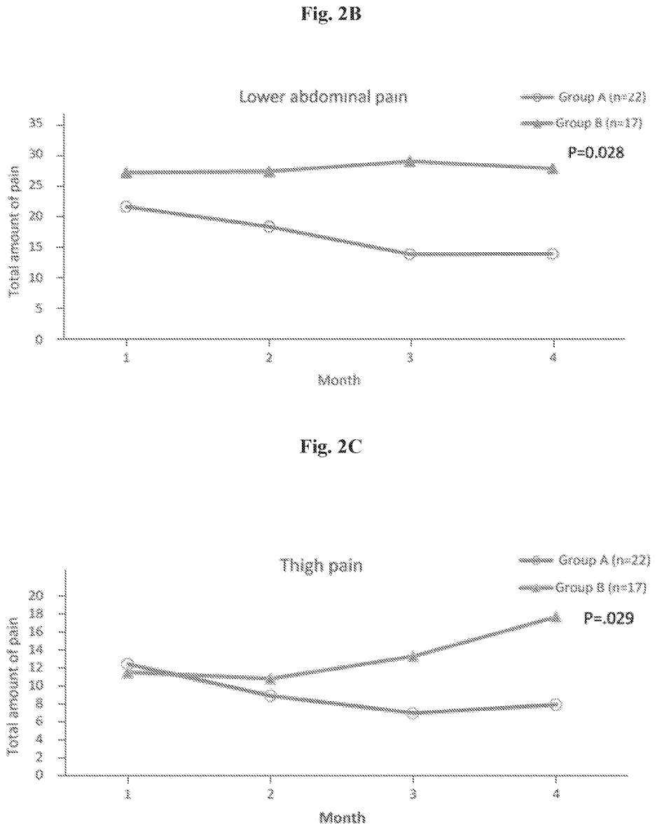

Weighted pain analysis showed a decrease in the total amount of pain for the study group vs. control group patients with regards to back pain (p=0.034), thigh pain (p=0.029) and lower abdominal pain (p=0.028) (FIGS. 2A-2C). For pelvic pain, a borderline difference was obtained (p=0.054).

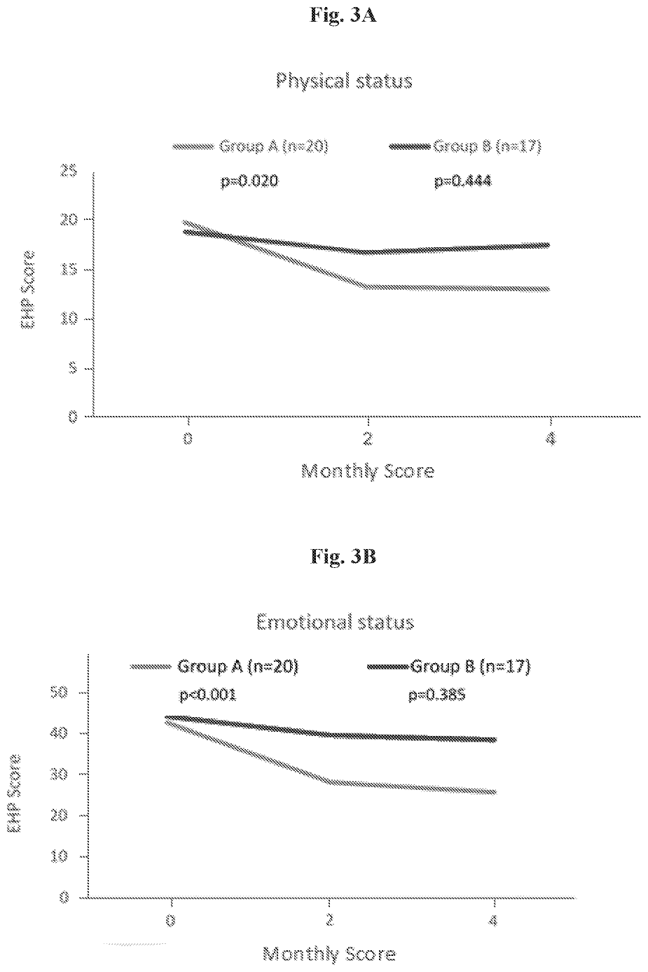

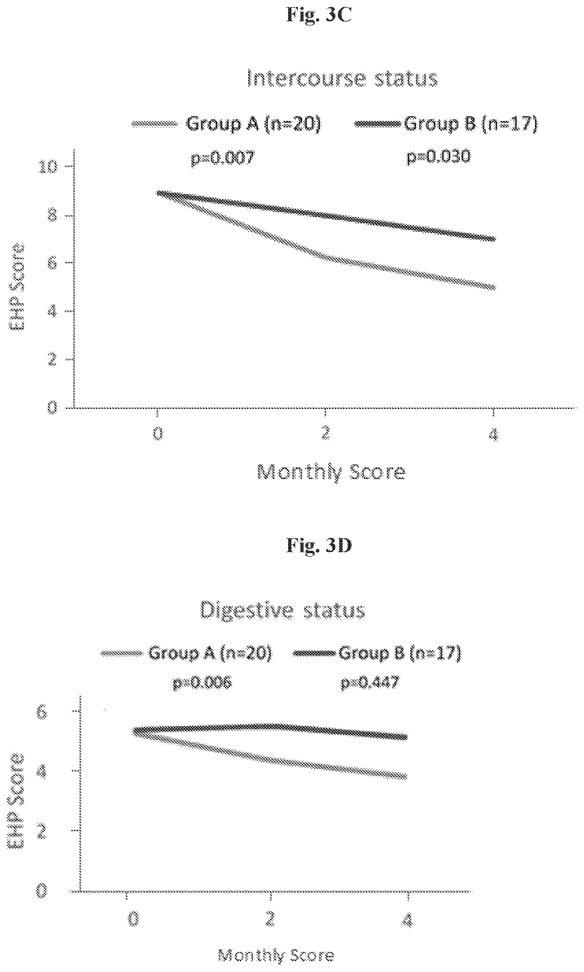

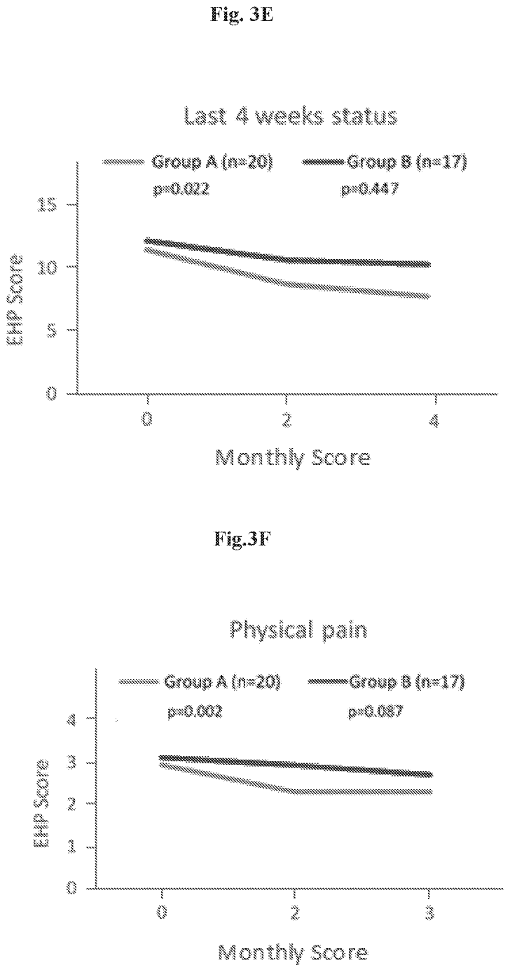

Analysis of the EHP-30 questionnaires showed significant improvement in quality of life for the study and control groups in all parameters of the questionnaire (p<0.05). Representative categories of life quality surveyed by the questionnaires are schematically in FIGS. 3A-3F.

1. A herbal formulation comprising at least one herb or a component thereof from three or more different classes of Chinese herbs selected from Blood tonifying, Blood moderators, Blood Heat coolers, body Heat coolers and Yang tonic herbs, wherein:
(i) the Blood tonifying herbs are selected from: Bai Shao, Dang Gui, Gou Qi Zi, He Shou Wu, Sang Shen Zi and Shu Di Huang;
(ii) the Blood moderating herbs are selected from Pu Huang, Yan Hu Suo, Hong Hua, San Leng, Dan Shen, Huai Niu Xi, Jiang Huang (curcumin), Ai Ye, Ce Bai Ye, Di Yu, Huai Hua Mi, Lian Fang, Ou Jie, Wang Bu Liu Xing, and Zong Lu Pi, Chi Shao, Chuan Niu Xi, Chuan Shan Jia, Chuan Xiong, Ding Jin Cao, E Zhu, Gan Qi, Hu Zhang, Ji Xue Teng, Liu Ji Nu, Ma Bian Cao, Qi Cao, Shui Zhi, Su Mu, Yi Mu Cao, Yu Jin, and Ze Lan;
(iii) the Blood or body Heat cooling herbs are selected from, Xia Ku Cao, Chai Hu, Mu Dan Pi, Cha Chi Huang, Ren Zhong Bai, Zhi Zi, Bo He, Man Ji Zhi, Mao ZI Dun Tou, Sang Ye, Yi Zhi Xiang, Di Gu Pi, Ji Guan Hua, Xi Jiao, Xuan Shen; Yin Chai Hu and Zi Cao; and
(iv) the Yang tonic herbs are selected from Xu Duan, Ba Ji Tian; Bu Gu Zhi; Du Zhong, Gou Ji, Hai Gou Shen, Hai Tao Ren; Jiu Zi; Rou Cong Rong, She Chuang Zi, Tu Si Zi and Xian Mao,
and equivalents thereof.

2. The herbal formulation of claim 1, wherein:
(i) the Blood tonifying herb is Bai Shao, Dang Gui or both;
(ii) the Blood moderating herbs are one or more of Pu Huang, Yan Hu Suo, Hong Hua, San Leng, Dan Shen, Huai Niu Xi and Jiang Huang (curcumin);
(iii) the Blood or body Heat cooling herbs are one or more of Xia Ku Cao, Chai Hu, and Mu Dan Pi and
(iv) the Yang tonic herb is Xu Duan.

3. The herbal formulation of claim 1, comprising three or more herbs selected from: Bai Shao, Dang Gui, E Zhu, Pu Huang, Yan Hu Suo, Hong Hua, San Leng, Yi Mu Cao, Dan Shen, Huai Niu Xi, Jiang Huang, Xia Ku Cao, Chai Hu, Mu Dan Pi or Xu Duan, Du Zhong, Rou Cong Rong, Xian Mao, Yi Zhi Ren or Ba Ji Tian, wherein at least three herbs belong to at least three different classes.

4. The herbal formulation of claim 1, comprising:
(i) San Leng in an amount of from 6.5% to 8% w/w;
(ii) Dan Shen in an amount of from 8.5% to 10% w/w;
(iii) Bai Shao in an amount of from 8.5% to 10% w/w;
(iv) Mu Dan Pi in an amount of from 8.5% to 10% w/w;
(v) Chai Hu in an amount of from 4.0% to 5.5% w/w;
(vi) Pu Huang in an amount of from 2.0% to 3.0% w/w; and
(vii) Xu Duan in an amount of from 6.0%-8.0% w/w.

5. The herbal formulation of claim 1, comprising:
(i) Pu Huang in an amount of from 2% to 5% w/w;
(ii) Yan Hu Suo in an amount of from 6% to 9% w/w;
(iii) Hong Hua in an amount of from 2% to 6% w/w;
(iv) San Leng in an amount of from 6% to 9% w/w;
(v) Xia Ku Cao in an amount of from 6% to 8% w/w;
(vi) Bai Shao in an amount of from 8% to 10% w/w;
(vii) Chai Hu in an amount of from 3% to 9% w/w;
(viii) Dan Shen in an amount of from 6% to 14% w/w;
(ix) Mu Dan Pi in an amount of from 8% to 12% w/w;
(x) Xu Duan in an amount of from 6% to 12% w/w;
(xi) Huai Niu Xi in an amount of from 6% to 10% w/w;
(xii) Jiang Huang in an amount of from 3% to 9% w/w; and
(xiii) Curcumin extract in an amount of from 10% to 20% w/w.

6. The herbal formulation of claim 1, further comprising Qing Dai in an amount of from 20% to 40% w/w.

7. The herbal formulation of claim 1, formulated as a solid or liquid dosage form.

8. The herbal formulation of claim 7, wherein the solid dosage form is at least one of a capsule, pill, caplet, powder or a tablet.

9. The herbal formulation of claim 1, formulated for oral administration.

10. The herbal formulation of claim 1, formulated as a single unit-dose dosage form.

11. The herbal formulation of claim 1, formulated as a multiple unit-dose dosage form.

12. A method for treating, preventing, alleviating and/or mitigating pain, comprising administrating to a subject in need thereof therapeutically effective amounts of one or more herbal formulations according claim 1, thereby treating, preventing, alleviating and/or mitigating pain in the subject.

13. A method for treating, preventing and/or alleviating Blood stagnation, comprising administrating to a subject in need thereof therapeutically effective amounts of one or more herbal formulations according to claim 1, thereby treating, preventing and/or alleviating Blood stagnation in the subject.

14. A method for treating, preventing and/or alleviating inflammatory and/or hormone mediated diseases, disorders and conditions, comprising administrating to a subject in need thereof therapeutically effective amounts of one or more herbal formulations according to claim 1, thereby treating, preventing and/or alleviating inflammatory and/or hormone mediated diseases, disorders and conditions in the subject.

15. A method for treating, preventing and/or alleviating inflammatory and/or hormone mediated diseases, disorders and conditions of the pelvis and lower abdomen, comprising administrating to a subject in need thereof therapeutically effective amounts of one or more herbal formulations according to claim 1, thereby treating, preventing and/or alleviating inflammatory and/or hormone mediated diseases, disorders and conditions of the pelvis and lower abdomen in the subject.

16. The method of claim 15, wherein the inflammatory and/or hormone mediated diseases, disorders and conditions of the pelvis and lower abdomen are selected from the group consisting of endometriosis, adenomyosis, chronic pelvic pain, painful bladder syndrome, interstitial cystitis, inflammatory bowel disease, irritable bowel syndrome, vulvodynia, vestibulodinia (vestibulitis), myofascial syndrome and associated morbidity including chronic fatigue syndrome, premenstrual syndrome (PMS) symptoms mood changes and catamenia hemothorax.

17. The method of claim 16, wherein the disease is endometriosis.
